(19) United States
(12) Patent Application Publication
(10) Pub. No.: US 2024/0269012 A1
(43) Pub. Date: Aug. 15, 2024

(54) ABSORBENT ARTICLES WITH BARRIER LEG CUFFS HAVING A BACKFOLDED INNER CUFF

(71) Applicant: The Procter & Gamble Company, Cincinnati, OH (US)

(72) Inventors: Abhishek SURUSHE, Mason, OH (US); Jeromy Thomas RAYCHECK, South Lebanon, OH (US)

(21) Appl. No.: 18/437,295

(22) Filed: Feb. 9, 2024

Related U.S. Application Data
(60) Provisional application No. 63/444,719, filed on Feb. 10, 2023.

(51) Int. Cl. A61F 13/49; A61F 13/15; A61F 13/475; A61F 13/494

(52) U.S. Cl. CPC A61F 13/49014; A61F 13/4752; A61F 2013/15422; A61F 2013/49025; A61F 2013/4944

(57) ABSTRACT

Absorbent articles having a liquid permeable topsheet, a liquid impermeable backsheet, an absorbent core disposed at least partially between the topsheet and the backsheet, and a pair of barrier leg cuffs is provided. Each barrier leg cuff is formed from a single web of material and has a backfolded inner cuff with an inner folded edge, a backfold folded edge, an inner elastic element, and a backfold elastic element. The inner elastic element may be disposed proximate the inner folded edge, and the backfold elastic element may be disposed proximate the backfold folded edge. At least a portion of the backfolded inner cuff may be configured to overlap the topsheet when the absorbent article is in a flat, uncontracted configuration.

### CROSS REFERENCE TO RELATED APPLICATION

This application claims the benefit, under 35 USC 119(e), to U.S. Provisional Patent Application No. 63/444,719 filed on Feb. 10, 2023, which is herein incorporated by reference in its entirety.

### FIELD

The present disclosure is generally directed to absorbent articles (e.g., diapers, adult incontinence articles, feminine hygiene pads, disposable absorbent inserts, and the like) having barrier leg cuffs with backfolded inner cuffs.

### BACKGROUND

Absorbent articles, such as diapers, adult incontinence articles, feminine hygiene pads, disposable absorbent inserts, and the like are often used for containing urine and/or other bodily exudates. To effectively contain bodily exudates, the articles should provide a snug fit around the waist and legs of a wearer.

Current absorbent article designs frequently include a barrier leg cuff to assist in the reduction of leakage of bodily exudates. Barrier leg cuffs typically include an inner cuff that is disposed inboard of the absorbent member of the absorbent article and is meant to contain bodily exudates within the absorbent article and proximate the absorbent member so it can be absorbed. The inner cuff may have an elastic strand disposed therein such that the inner cuff may form a gasket along the leg of the wearer to help inhibit bodily exudates from leaking out of the absorbent article. These current designs, however, may fail to form a gasket completely around the leg of a wearer, especially when the wearer is moving about. This is due to the inner cuff forming a gasket by a single touch point with the legs of the wearer, typically at a terminal end of the inner cuff. When the wearer changes positions or moves their leg, a portion of the inner cuff may lose contact with the leg of the wearer, thus allowing for leakage of bodily exudates.

Furthermore, inner cuffs are generally designed such that the elastic is disposed only at the terminal end of the inner cuff where the cuff contacts the leg. The placement of the elastic only at the terminal end of the inner cuff thus defines the height of the cuff, and therefore the void volume—the volume made available when the absorbent article is placed on the wearer—available to contain bodily exudates. Such a configuration can limit the void volume because the height of the inner cuffs may be limited in order to be sure they form a proper gasket with the leg of the wearer. Greater void volumes are desirable in order to manage greater volumes of bodily exudates and/or allow a wearer to use a single absorbent article for a longer time, such as overnight.

In view of the problems with current barrier leg cuff designs and functionality, absorbent articles with barrier leg cuffs should be improved.

### SUMMARY

Aspects of the present disclosure solve one or more of the problems discussed above by providing an absorbent article comprising a pair of barrier leg cuffs, each barrier leg cuff comprising a backfolded inner cuff. The barrier leg cuff comprising a backfolded inner cuff creates a double seal around a wearer's leg, resulting in reduced gaps and reduced leakage. The barrier leg cuff comprising a backfolded inner cuff may also cause a broad portion of the backfolded inner cuff to rest against the leg of a wearer, resulting in a wider barrier against leakage. Furthermore, the barrier leg cuff comprising a backfolded inner cuff may allow for absorbent articles with larger void volumes than are presently available.

The present disclosure provides, in part, an absorbent article comprising a liquid permeable topsheet, a liquid impermeable backsheet, an absorbent core disposed at least partially between the topsheet and the backsheet, and a pair of barrier leg cuffs. Each barrier leg cuff is formed from a single web of material and comprises a backfolded inner cuff comprising an inner folded edge, a backfold folded edge, an inner elastic element, and a backfold elastic element. The inner elastic element may be disposed proximate the inner folded edge, and the backfold elastic element may be disposed proximate the backfold folded edge. At least a portion of the backfolded inner cuff may be configured to overlap the topsheet when the absorbent article is in a flat, uncontracted configuration.

The present disclosure provides, in part, an absorbent article comprising a chassis comprising a liquid permeable topsheet, a liquid impermeable backsheet, and an absorbent core disposed at least partially between the topsheet and the backsheet. The absorbent article further comprises a pair of barrier leg cuffs, each barrier leg cuff formed from a single web of material and comprising a proximal portion joined to the chassis and an inner distal portion. The web of material is folded laterally outward to form a backfold between the proximal portion and the inner distal portion, forming a backfolded inner cuff. The backfolded inner cuff comprises an inner folded edge, a backfold folded edge, an inner elastic element, and a backfold elastic element.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is a plan view of an example absorbent article in the form of a taped diaper, garment-facing surface facing the viewer, in a flat laid-out state;

FIG. 2 is a plan view of the example absorbent article of FIG. 1, wearer-facing surface facing the viewer, in a flat laid-out state;

FIG. 3 is a front perspective view of the absorbent article of FIGS. 1 and 2 in a fastened position;

FIG. 4 is a front perspective view of an absorbent article in the form of a pant;

FIG. 5 is a rear perspective view of the absorbent article of FIG. 4;

FIG. 6 is a plan view of the absorbent article of FIG. 4, laid flat, with a garment-facing surface facing the viewer;

FIG. 7 is a cross-sectional view of the absorbent article taken about line 7-7 of FIG. 6;

FIG. 8 is a cross-sectional view of the absorbent article taken about line 8-8 of FIG. 6;

FIG. 9 is a plan view of an example absorbent core or an absorbent article;

FIG. 10 is a cross-sectional view, taken about line 10-10, of the absorbent core of FIG. 9;

FIG. 11 is a cross-sectional view, taken about line 11-11, of the absorbent core of FIG. 10;

FIG. 12 is a plan view of an example absorbent article of the present disclosure that is a sanitary napkin;

FIG. 13 is a schematic cross-sectional view of a barrier leg cuff having a backfolded inner cuff;

FIG. 14 is a schematic cross-sectional view of a barrier leg cuff having a backfolded inner cuff comprising a plurality of inner and backfold elastic members;

FIG. 15 is a plan view of an example absorbent article in the form of a taped diaper with barrier leg cuffs having backfolded inner cuffs and outer cuffs, wearer-facing surface facing the viewer, in a flat laid-out state;

FIG. 16 is a schematic cross-sectional view of a barrier leg cuff having a backfolded inner cuff having a single inner elastic member and a single backfold elastic member;

FIG. 17 is a schematic cross-sectional view of a barrier leg cuff having a backfolded inner cuff having a plurality of inner elastic members and a single backfold elastic member;

FIG. 18 is a schematic cross-sectional view of a barrier leg cuff having a backfolded inner cuff having a single inner elastic member and a plurality of backfold elastic members;

FIG. 19 is a schematic cross-sectional view of a barrier leg cuff having a backfolded inner cuff taken about line 19-19 of FIG. 15;

FIG. 20 is a schematic cross-sectional view of a barrier leg cuff having a backfolded inner cuff taken about line 20-20 of FIG. 15;

FIG. 21 is a schematic cross-sectional view of a barrier leg cuff having a backfolded inner cuff and an outer cuffs;

FIG. 22 is a schematic cross-sectional view of a barrier leg cuff having a backfolded inner cuff and an outer cuffs, where the outer cuff elastic element is disposed between the web of material forming the barrier leg cuff and the backsheet and/or outer cover;

FIGS. 23A-F are schematic cross-sectional views of various stretch laminates;

FIG. 24 is an exploded perspective view of an exemplary ear comprising a stretch laminate and illustrating exemplary surface modifications;

FIG. 25 is a schematic perspective view of an absorbent article in the form of an insert and a reusable outer cover;

FIG. 26 is an example cross-sectional view of a nonwoven material forming the barrier leg cuff; and

FIG. 27 is a further example cross-sectional view of a nonwoven material forming the barrier leg cuff.

### DETAILED DESCRIPTION

Various non-limiting forms of the present disclosure will now be described to provide an overall understanding of the principles of the structure, function, manufacture, and use of the absorbent articles with barrier leg cuffs having a backfolded inner cuff disclosed herein. One or more examples of these non-limiting forms are illustrated in the accompanying drawings. Those of ordinary skill in the art will understand that the absorbent articles with barrier leg cuffs having a backfolded inner cuff described herein and illustrated in the accompanying drawings are non-limiting example forms. The features illustrated or described in connection with one non-limiting form may be combined with the features of other non-limiting forms. Such modifications and variations are intended to be included within the scope of the present disclosure.

### General Description of an Absorbent Article

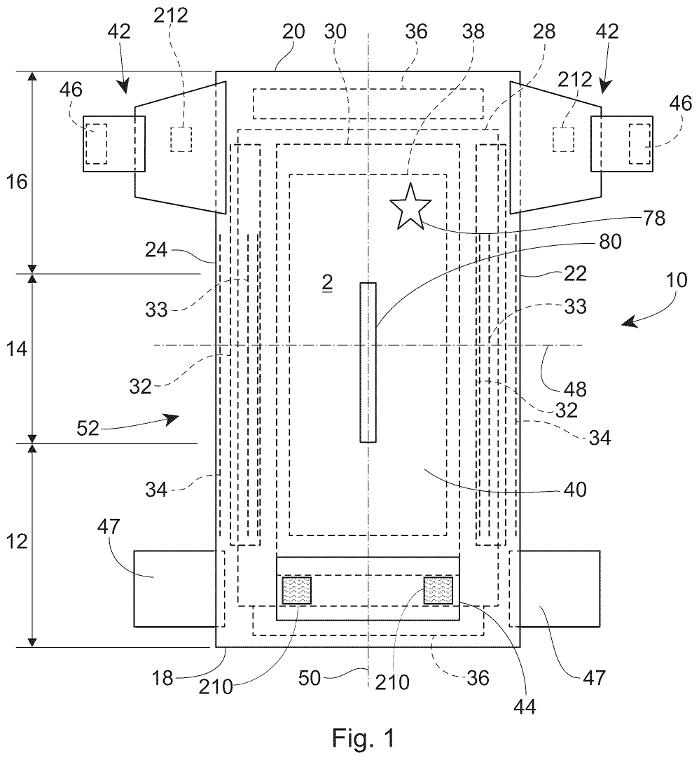

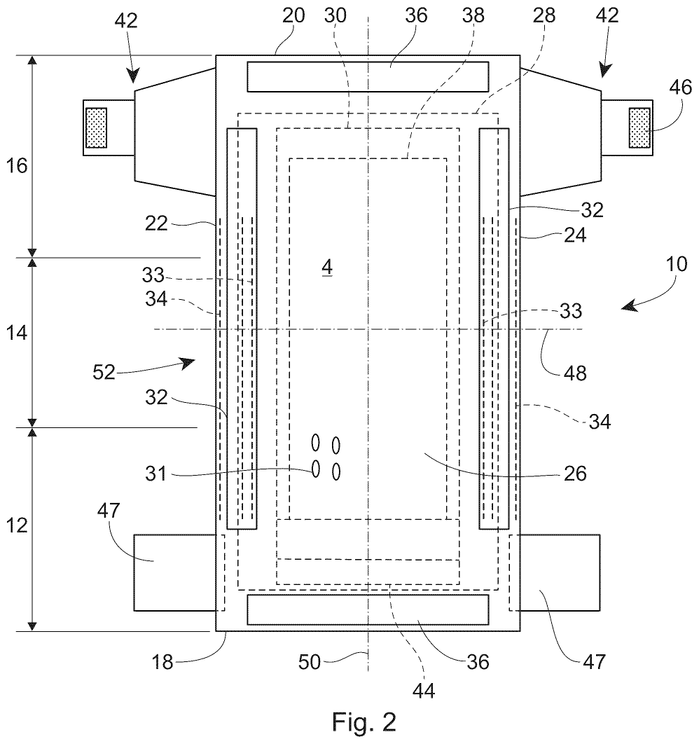

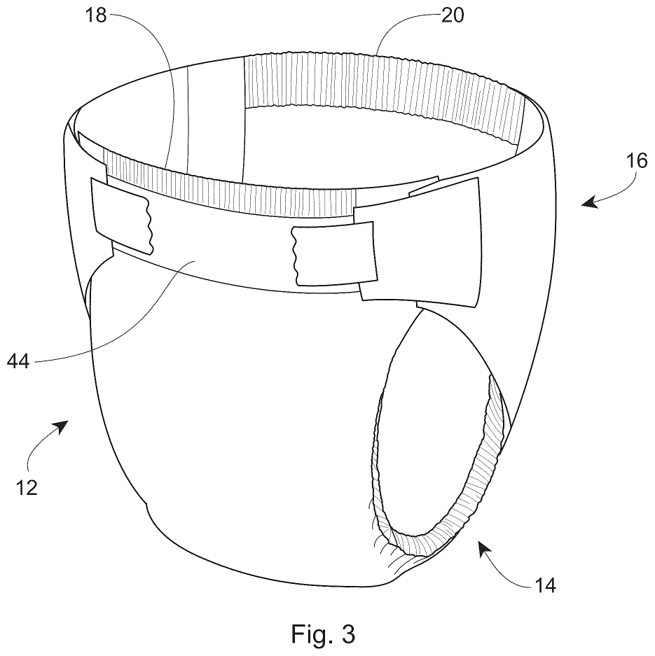

An example absorbent article 10 according to the present disclosure, shown in the form of a taped diaper, is represented in FIGS. 1-3. FIG. 1 is a plan view of the example absorbent article 10, garment-facing surface 2 facing the viewer in a flat, laid-out state (i.e., no elastic contraction). FIG. 2 is a plan view of the example absorbent article 10 of FIG. 1, wearer-facing surface 4 facing the viewer in a flat, laid-out state. FIG. 3 is a front perspective view of the absorbent article 10 of FIGS. 1 and 2 in a fastened configuration. The absorbent article 10 of FIGS. 1-3 is shown for illustration purposes only as the present disclosure may be used for making a wide variety of absorbent articles, including diapers, adult incontinence articles, pant-style articles, sanitary napkins, and absorbent inserts, for example.

The absorbent article 10 may comprise a front waist region 12, a crotch region 14, and a back waist region 16. The crotch region 14 may extend intermediate the front waist region 12 and the back waist region 16. The front wait region 12, the crotch region 14, and the back waist region 16 may each be approximately ⅓ of the length of the absorbent article 10. The absorbent article 10 may comprise a front end edge 18, a back end edge 20 opposite to the front end edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially between the topsheet 26 and the backsheet 28. The absorbent article 10 may also comprise one or more pairs of barrier leg cuffs 32 as described further herein, one or more elastic waistbands 36, and/or one or more acquisition materials 38. The acquisition material or materials 38 may be positioned between the topsheet 26 and the absorbent core 30. An outer cover material 40, such as a nonwoven material, may cover a garment-facing side of the backsheet 28. The absorbent article 10 may comprise back ears 42 in the back waist region 16. The back ears 42 may comprise fasteners 46 and may extend from the back waist region 16 of the absorbent article 10 and attach (using the fasteners 46) to the landing zone area or landing zone material 44 on a garment-facing portion of the front waist region 12 of the absorbent article 10. The absorbent article 10 may also have front ears 47 in the front waist region 12. The absorbent article 10 may have a central lateral (or transverse) axis 48 and a central longitudinal axis 50. The central lateral axis 48 extends perpendicular to the central longitudinal axis 50.

The absorbent article may comprise a secondary fastening system comprising a secondary fastening component 210 and a secondary receiving component 212 that are operatively engageable to further secure the article about the wearer. The secondary fastening component 210 may be disposed in the front waist region 12, and the secondary f receiving component 212 may be disposed in the back waist region 16. Addition of a secondary fastening system can provide a greater surface area for fastening, and thereby de-concentrate lateral tensile forces communicated through the fastening location(s) as the rear waist region is pulled toward the front waist region, and vice versa, when the diaper is worn. In addition, having two distinct fastening locations reduces the tendency of the front portion of the article to pivot (i.e., pivot around the single fastening location of the primary fastening system). Further, the secondary system helps to create a line of tension closer to the front waist edge, which may reduce the likelihood of folding or flipping over of the front waist edge during wear. Further still, the secondary system may create an anchoring geodesic to direct forces from the crotch region to over the hips in order to prevent sagging during wearer. The secondary system may also help to secure the front ear or combination belt structures in place during wear. Each of the foregoing can serve to provide for more effective and durable fastening and less longitudinal and/or lateral flexing, sagging and/or wrinkling of the diaper materials about the fastening areas during wear.

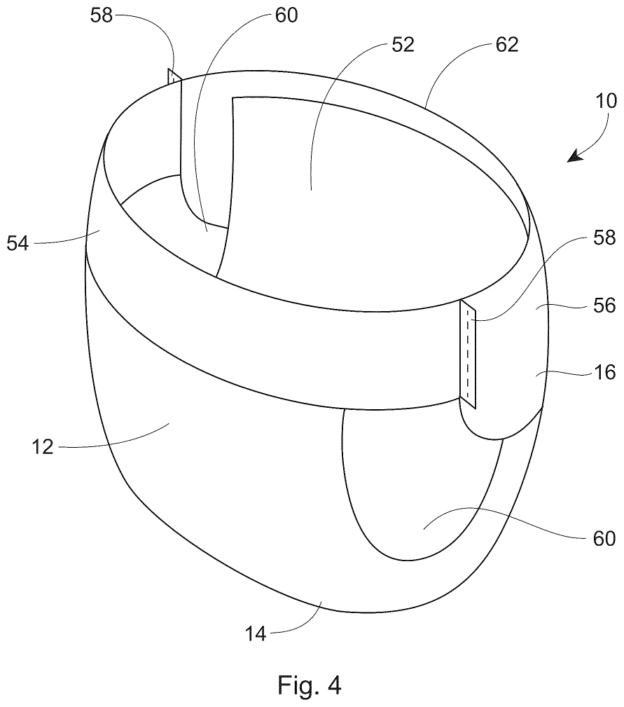

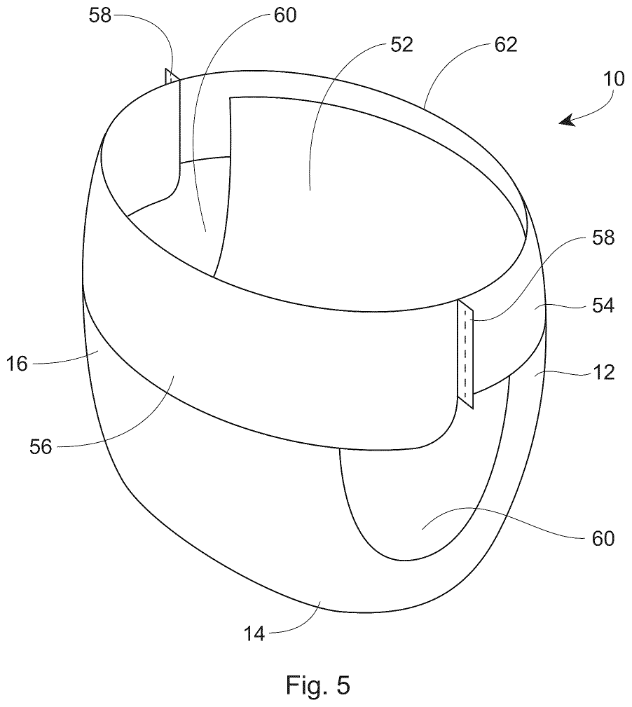

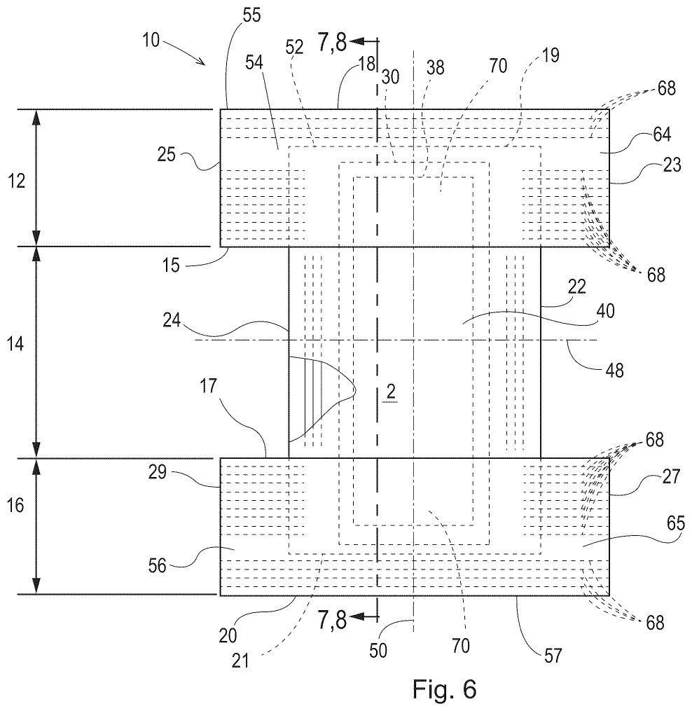

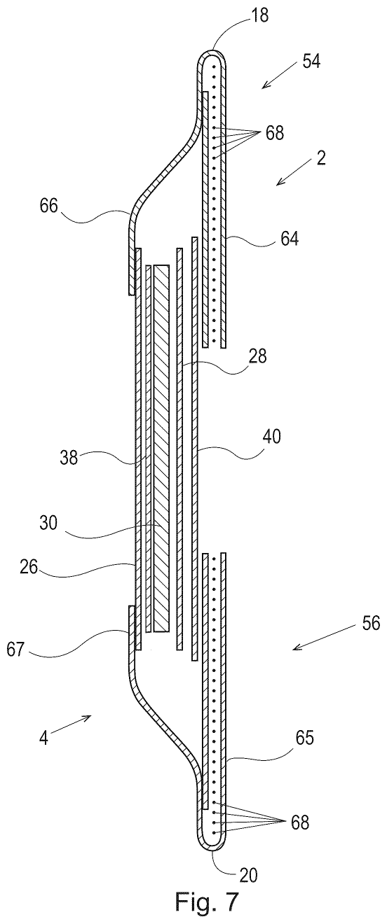

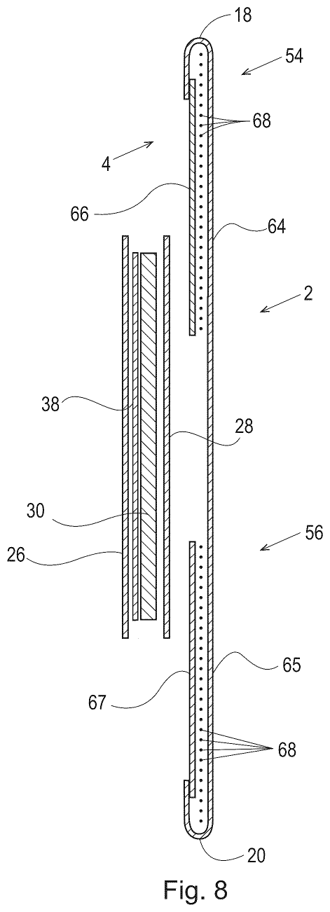

In other instances, the absorbent article may be in the form of a pant (pant-style) having permanent or refastenable side seams. Suitable refastenable seams are disclosed in U.S. Pat. Appl. Pub. No. 2014/0005020 and U.S. Pat. No. 9,421,137. Referring to FIGS. 4-8, an example absorbent article 10 in the form of a pant is illustrated. FIG. 4 is a front perspective view of the absorbent article 10. FIG. 5 is a rear perspective view of the absorbent article 10. FIG. 6 is a plan view of the absorbent article 10, laid flat, with the garment-facing surface facing the viewer. Elements of FIG. 4-8 having the same reference number as described above with respect to FIGS. 1-3 may be the same element (e.g., absorbent core 30). FIG. 7 is an example cross-sectional view of the absorbent article taken about line 7-7 of FIG. 6. FIG. 8 is an example cross-sectional view of the absorbent article taken about line 8-8 of FIG. 6. FIGS. 7 and 8 illustrate example forms of front and back belts 54, 56. The absorbent article 10 may have a front waist region 12, a crotch region 14, and a back waist region 16. Each of the regions 12, 14, and 16 may be ⅓ of the length of the absorbent article 10. The absorbent article 10 may have a chassis 52 (sometimes referred to as a central chassis or central panel) comprising a topsheet 26, a backsheet 28, and an absorbent core 30 disposed at least partially intermediate the topsheet 26 and the backsheet 28, and an optional acquisition material 38, similar to that as described above with respect to FIGS. 1-3. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges 23 and 25 of the front belt 54 may be joined to side edges 27 and 29, respectively, of the back belt 56 to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

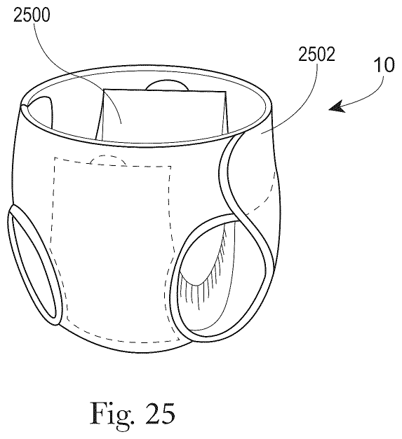

In another form, the absorbent article may be an insert 2500 for use with a reusable outer cover 2502, as shown in FIG. 25. The insert 2500 may be disposable or reusable. The reusable outer cover 2502 may comprise a woven or other material and may be configured as a pant or a taped diaper. In the taped context, the reusable outer cover 2502 may comprise a fastening system used to join a front waist region of the reusable outer cover to a back waist region. The fastening system may comprise snaps, buttons, and/or hooks and loops, for example. The insert 2500 may comprise a liquid permeable topsheet, a liquid impermeable backsheet, and an absorbent core positioned at least partially between the topsheet and the backsheet. One or more acquisition and/or distribution materials may be positioned between the topsheet and the absorbent core. The insert 2500 may comprise one or more pairs of leg cuffs and may be free of ears, side panels, and/or waistbands. In some instances, a nonwoven material may be positioned on a garment-facing side of the backsheet. A garment-facing surface of the insert 2500 may be attached to a wearer-facing surface of the reusable outer cover 2502 via adhesives, hook and loop fasteners, or other methods of joinder. An example insert and reusable outer cover system is disclosed in U.S. Pat. No. 9,011,402, issued on Apr. 21, 2015, to Roe, et al. The insert or the reusable outer cover may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Belts

Referring to FIGS. 7 and 8, the front and back belts 54 and 56 may comprise front and back inner belt layers 66 and 67 and front and back outer belt layers 64 and 65 having an elastomeric material (e.g., strands 68 or a film (which may be apertured)) disposed at least partially therebetween. The elastic elements 68 or the film may be relaxed (including being cut) to reduce elastic strain over the absorbent core 30 or may alternatively run continuously across the absorbent core 30. The elastics elements 68 may have uniform or variable spacing therebetween in any portion of the belts. The elastic elements 68 may also be pre-strained the same amount or different amounts. The front and/or back belts 54 and 56 may have one or more elastic element free zones 70 where the chassis 52 overlaps the belts 54, 56. In other instances, at least some of the elastic elements 68 may extend continuously across the chassis 52.

The front and back inner belt layers 66, 67 and the front and back outer belt layers 64, 65 may be joined using adhesives, heat bonds, pressure bonds or thermoplastic bonds. Various suitable belt layer configurations can be found in U.S. Pat. Appl. Pub. No. 2013/0211363.

Front and back belt end edges 55 and 57 may extend longitudinally beyond the front and back chassis end edges 19 and 21 (as shown in FIG. 6) or they may be co-terminus. The front and back belt side edges 23, 25, 27, and 29 may extend laterally beyond the chassis side edges 22 and 24. The front and back belts 54 and 56 may be continuous (i.e., having at least one layer that is continuous) from belt side edge to belt side edge (e.g., the transverse distances from 23 to 25 and from 27 to 29). Alternatively, the front and back belts 54 and 56 may be discontinuous from belt side edge to belt side edge (e.g., the transverse distances from 23 to 25 and 27 to 29), such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side seams 58.

The front outer belt layer 64 and the back outer belt layer 65 may be separated from each other, such that the layers are discrete or, alternatively, these layers may be continuous, such that a layer runs continuously from the front belt end edge 55 to the back belt end edge 57. This may also be true for the front and back inner belt layers 66 and 67—that is, they may also be longitudinally discrete or continuous. Further, the front and back outer belt layers 64 and 65 may be longitudinally continuous while the front and back inner belt layers 66 and 67 are longitudinally discrete, such that a gap is formed between them—a gap between the front and back inner and outer belt layers 64, 65, 66, and 67 is shown in FIG. 7 and a gap between the front and back inner belt layers 66 and 67 is shown in FIG. 8.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 4 and 5).

The front and back belts 54 and 56 may comprise graphics (see e.g., 78 of FIG. 1). The graphics may extend substantially around the entire circumference of the absorbent article 10 and may be disposed across side seams 58 and/or across proximal front and back belt seams 15 and 17; or, alternatively, adjacent to the seams 58, 15, and 17 in the manner described in U.S. Pat. No. 9,498, 389 to create a more underwear-like article. The graphics may also be discontinuous.

Alternatively, instead of attaching belts 54 and 56 to the chassis 52 to form a pant, discrete side panels may be attached to side edges of the chassis 22 and 24. Suitable forms of pants comprising discrete side panels are disclosed in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

### Topsheet

The topsheet 26 is the part of the absorbent article 10 that is in contact with the wearer's skin. The topsheet 26 may be joined to portions of the backsheet 28, the absorbent core 30, the barrier leg cuffs 32, and/or any other layers as is known to those of ordinary skill in the art. The topsheet 26 may be compliant, soft-feeling, and non-irritating to the wearer's skin. Further, at least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet may have one or more layers. The topsheet may be apertured (FIG. 2, element 31), may have any suitable three-dimensional features, and/or may have a plurality of embossments (e.g., a bond pattern). The topsheet may be apertured by overbonding a material and then rupturing the overbonds through ring rolling, such as disclosed in U.S. Pat. No. 5,628,097, to Benson et al., issued on May 13, 1997 and disclosed in U.S. Pat. Appl. Publication No. US 2016/0136014 to Arora et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet. The topsheet may comprise a one, or more, layer hydroentangled material with or without apertures. The topsheet may comprise a variable basis weight nonwoven material.

### Backsheet

The backsheet 28 is generally that portion of the absorbent article 10 positioned proximate to the garment-facing surface of the absorbent core 30. The backsheet 28 may be joined to portions of the topsheet 26, the outer cover material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials which permit vapors to escape from the absorbent article, while still preventing, or at least inhibiting, bodily exudates from passing through the backsheet.

### Outer Cover Material

The outer cover material (sometimes referred to as a backsheet nonwoven) 40 may comprise one or more nonwoven materials joined to the backsheet 28 and that covers the backsheet 28. The outer cover material 40 forms at least a portion of the garment-facing surface 2 of the absorbent article 10 and effectively “covers” the backsheet 28 so that film is not present on the garment-facing surface 2. The outer cover material 40 may comprise a bond pattern, apertures, and/or three-dimensional features. The outer cover material 40 may be a hydroentangled nonwoven material.

### Absorbent Core

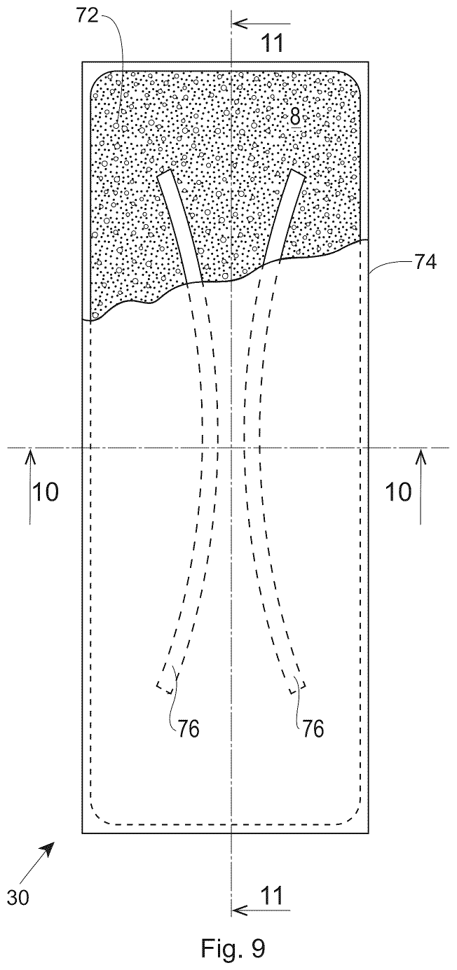

As used herein, the term “absorbent core” 30 refers to a component of the absorbent article 10 disposed in the article for absorbing and containing liquid such as urine received by the absorbent article. The absorbent core thus typically has a high absorbent capacity. An example absorbent core 30 is schematically shown in FIGS. 9-11. The absorbent core comprises an absorbent material 72, that is typically enclosed within or sandwiched between a core bag 74.

The core wrap may be a single material that is folded and attached to itself, or it may comprise a separate top layer and bottom layer that may be bonded or otherwise joined together. The absorbent material typically comprises superabsorbent particles which are optionally mixed with cellulose fibers. As used herein, “absorbent core” does not include any acquisition-distribution systems, topsheet, or backsheet of the absorbent article.

The example absorbent core 30 shown in isolation in FIGS. 9-11 is in the dry state (before use). The absorbent core may typically have a generally rectangular shape as defined by its longitudinal edges and transversal front edge and back edge or may have other shapes.

Absorbent material 72 may be deposited as an absorbent layer having a generally rectangular outline, as represented in FIG. 9. A wide variety of absorbent cores may also be used. The absorbent material 72 layer may also have a non-rectangular perimeter (“shaped” core), in particular, the absorbent material 72 may define a tapering along its width towards the central region of the core (or “dog-bone” shape). In this way, the absorbent material deposition area may have a relatively narrow width in an area of the core intended to be placed in the crotch region of the absorbent article. This may provide for example better wearing comfort. Other shapes can also be used such as a “T” or “Y” or “hourglass” for the area of the absorbent material.

The absorbent material 72 may be any conventional absorbent material known in the art. For example, the absorbent material may comprise a blend of cellulose fibers and superabsorbent particles (“SAP”), typically with the percentage of SAP ranging from about 50% to about 75% by weight of the absorbent material. The absorbent material may also be free of cellulose fibers, as is known in so-called airfelt-free cores, where the absorbent material consists, or consists essentially, of SAP. The absorbent material may also be a high internal phase emulsion foam

“Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test (EDANA method WSP 241.2.R3 (12)). The SAP may in particular have a CRC value of at least 20 g/g, in particular of from 20 g/g to 40 g/g. “Superabsorbent polymer particles”, as used herein, refers to a superabsorbent polymer material which is in particulate form so as to be flowable in the dry state.

Various absorbent core designs comprising high amounts of SAP have been proposed in the past, see for example in U.S. Pat. No. 5,599,335 (Goldman), EP1,447,066 (Busam), WO95/11652 (Tanzer), U.S. Pat. Appl. Pub. No. 2008/0312622A1 (Hundorf), WO2012/052172 (Van Malderen). In particular, the SAP printing technology as disclosed in U.S. Pat. Appl. Pub. No. 2006/024433 (Blessing), U.S. Pat. Appl. Pub. No. 2008/0312617 and U.S. Pat. Appl. Pub. No. 2010/0051166A1 (both to Hundorf et al.) may be used. The present disclosure however is not limited to a particular type of absorbent core. The absorbent core may also comprise one or more glues such as an auxiliary glue applied between the internal surface of one (or both) of the core wrap layers and the absorbent material to reduce leakage of SAP outside the core wrap. A micro-fibrous adhesive net may also be used in air-felt free cores as described in the above Hundorf references. These glues are not represented in the Figures for simplicity. Other core constructions comprising a high loft nonwoven substrate such as a carded nonwoven layer, having a porous structure into which SAP particles have been deposited, may also be used in present disclosure.

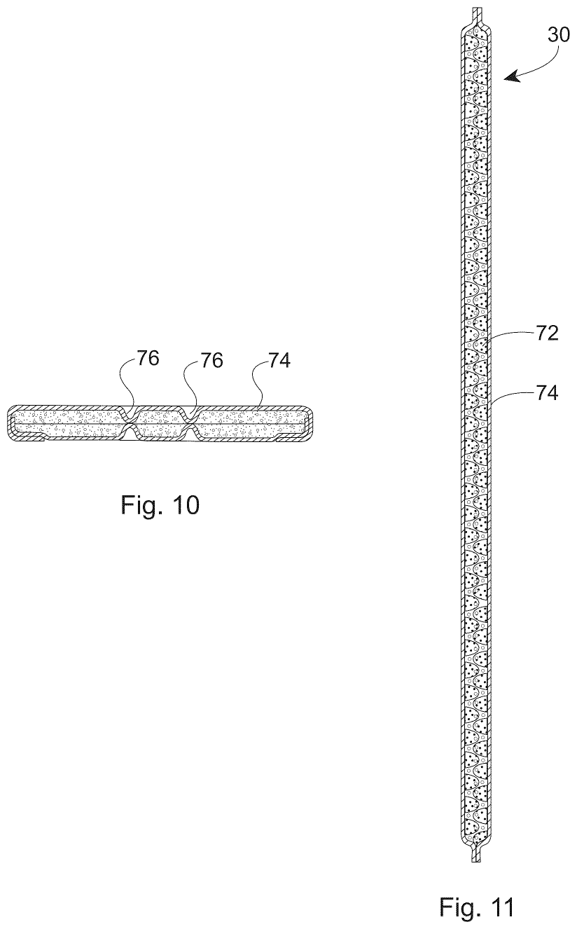

The absorbent material may be deposited as a continuous layer within the core wrap. The absorbent material may also be present discontinuously, for example, as individual pockets or stripes of absorbent material enclosed within the core wrap and separated from each other by material-free junction areas. A continuous layer of absorbent material, in particular of SAP, may also be obtained by combining two absorbent layers having matching discontinuous absorbent material application pattern, wherein the resulting layer is substantially continuously distributed across the absorbent particulate polymer material area, as illustrated in FIGS. 10-11. As for example taught in U.S. Pat. Appl. Pub. No. 2008/312,622 A1 (Hundorf), each absorbent material layer may thus comprise a pattern having absorbent material land areas and absorbent material-free junction areas, wherein the absorbent material land areas of the first layer correspond substantially to the absorbent material-free junction areas of the second layer and vice versa.

The basis weight (amount deposited per unit of surface) of the absorbent material may also be varied to create a profiled distribution of absorbent material, in particular in the longitudinal direction to provide more absorbency towards the center and the middle of the core, but also in the transversal direction, or both directions of the core. The absorbent core may also comprise one or more longitudinally (or otherwise) extending channels 76, which are areas of the absorbent layer substantially free of absorbent material within the absorbent material layer. The top side of the core wrap may be advantageously bonded to the bottom side of the core by adhesive, mechanical or ultra-sonic bonding through these material-free areas. Example disclosures of such channels in an airfelt-free core can be found in WO2012/170778 (Rosati et al.) and US2012/0312491 (Jackels). Channels may of course also be formed in absorbent cores comprising a mix of cellulose fibers and SAP particles. These channels may embody any suitable shapes and any suitable number of channels may be provided. In other instances, the absorbent core may be embossed to create the impression of channels. The absorbent core in FIGS. 9-11 is merely an example absorbent core. Many other absorbent cores with or without channels are also within the scope of the present disclosure.

### Elastic Waist Feature

Referring to FIGS. 1 and 2, the absorbent article 10 may comprise one or more elastic waist features 36. The one or more elastic waist features 36 may comprise a waistband or a waist cuff. The elastic waist feature 36 may be positioned on the garment-facing surface 2 or the wearer-facing surface 4. Alternatively, the elastic waist feature 36 may be positioned between the topsheet 26 and the backsheet 28. As an example, a first elastic waist feature 36 may be present in the front waist region 12 near the front waist edge 18 and a second elastic waist feature 36 may be present in the back waist region 16 near the back waist edge 20. The elastic waist feature 36 may aid in sealing the absorbent article 10 around a waist of a wearer and at least inhibiting bodily exudates from escaping the absorbent article 10 through the waist opening circumference. In some instances, an elastic waist feature may fully surround the waist opening circumference of an absorbent article 10. The elastic waist feature 36 may comprise an elastic film joined to the topsheet 26 and a nonwoven material covering the elastic film. In other instances, the elastic waist feature 36 may comprise an elastic film sandwiched between two nonwoven materials. The elastic film may be ultrasonically bonded, or otherwise bonded or attached, to the one or more nonwoven materials. The one or more nonwoven materials may be hydroentangled. The elastic film and/or the nonwoven materials may be preactivated (i.e., activated prior to being joined together) or the formed elastic film/nonwoven laminate may be activated post laminate formation.

### Acquisition Materials

Referring to FIGS. 1, 2, 7, and 8, one or more acquisition materials 38 may be present at least partially intermediate the topsheet 26 and the absorbent core 30. The acquisition materials 38 are typically hydrophilic materials that provide significant wicking of bodily exudates. These materials may dewater the topsheet 26 and quickly move bodily exudates into the absorbent core 30. The acquisition materials 38 may comprise one or more nonwoven materials, foams, formed films, apertured formed films, cellulosic materials, cross-linked cellulosic materials, air laid cellulosic nonwoven materials, spunlace materials, or combinations thereof, for example. In some instances, portions of the acquisition materials 38 may extend through portions of the topsheet 26, portions of the topsheet 26 may extend through portions of the acquisition materials 38, and/or the topsheet 26 may be nested with the acquisition materials 38. Typically, an acquisition material 38 may have a width and length that are smaller than the width and length of the topsheet 26. The acquisition material may be a secondary topsheet in the feminine pad context. The acquisition material may have one or more channels as described above with reference to the absorbent core 30 (including the embossed version). The channels in the acquisition material may align or not align with channels in the absorbent core 30. In an example, a first acquisition material may comprise a nonwoven material and as second acquisition material may comprise a cross-linked cellulosic material.

### Landing Zone

Referring to FIGS. 1 and 2, the absorbent article 10 may have a landing zone area 44 that is formed in a portion of the garment-facing surface 2 of the outer cover material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa.

### Wetness Indicator/Graphics

Referring to FIG. 1, the absorbent articles 10 of the present disclosure may comprise graphics 78 and/or wetness indicators 80 that are visible from the garment-facing surface 2. The graphics 78 may be printed on the landing zone 40, the backsheet 28, and/or at other locations. The wetness indicators 80 are typically applied to the absorbent core facing side of the backsheet 28, so that they can be contacted by bodily exudates within the absorbent core 30. In some instances, the wetness indicators 80 may form portions of the graphics 78. For example, a wetness indicator may appear or disappear and create/remove a character within some graphics. In other instances, the wetness indicators 80 may coordinate (e.g., same design, same pattern, same color) or not coordinate with the graphics 78.

### Front and Back Ears

Referring to FIGS. 1 and 2, as referenced above, the absorbent article 10 may have front and/or back ears 47, 42 in a taped diaper context. Only one set of ears may be required in most taped diapers. The single set of ears may comprise fasteners 46 configured to engage the landing zone or landing zone area 44. If two sets of ears are provided, in most instances, only one set of the ears may have fasteners 46, with the other set being free of fasteners. The ears, or portions thereof, may be elastic or may have elastic panels. In an example, an elastic film or elastic strands may be positioned intermediate a first nonwoven material and a second nonwoven material. The elastic film may or may not be apertured. The ears may be shaped. The ears may be integral (e.g., extension of the outer cover material 40, the backsheet 28, and/or the topsheet 26) or may be discrete components attached to a chassis 52 of the absorbent article on a wearer-facing surface 4, on the garment-facing surface 2, or intermediate the two surfaces 4, 2.

### Masking Layer

One or more masking layers or materials may be provided in the absorbent articles 10. A masking layer may be a layer that provides a cushiony feel when the absorbent article is touched from the garment-facing surface 2 or the wearer-facing surface 4. The masking layer may “mask” a grainy feel potentially caused by the absorbent material 72, such as superabsorbent polymers. The masking layer may “mask” bodily exudates from being visible when viewing the wearer-facing surface 4 or the garment-facing surface 2 of the absorbent article 10. The masking layer may have a basis weight in the range of about 15 gsm to about 50 gsm or about 15 gsm to about 40 gsm. The masking layer may comprise one or more nonwoven materials (e.g., a hydroentangled nonwoven material), foams, pulp layers, and/or other suitable materials. The masking layer may be the outer cover material 40. The masking layer may be the layer forming the garment-facing side or the wearer-facing side of the core bag 74. The masking layer may be a separate material positioned intermediate the garment-facing side of the core bag 74 and the liquid impermeable backsheet 28.

### Stretch Laminate

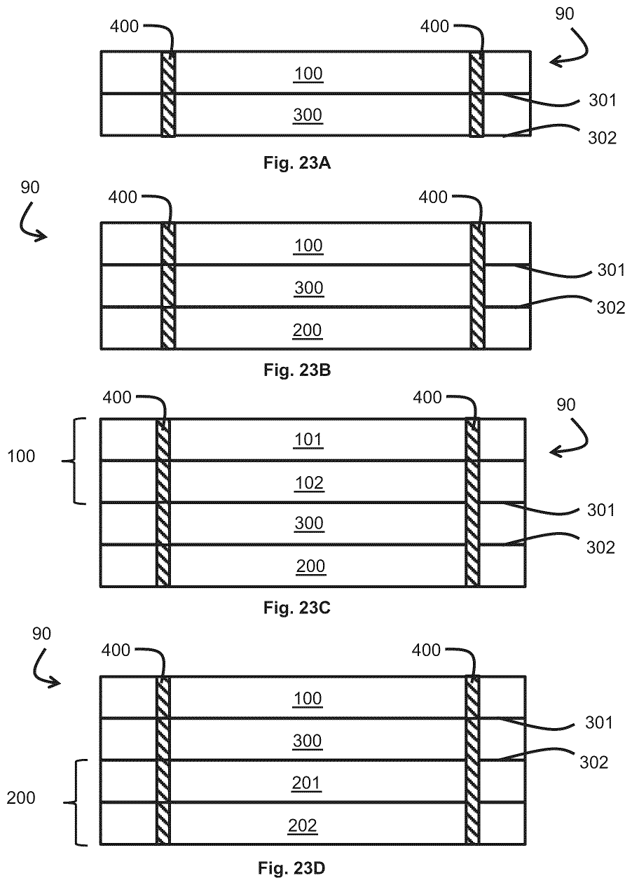

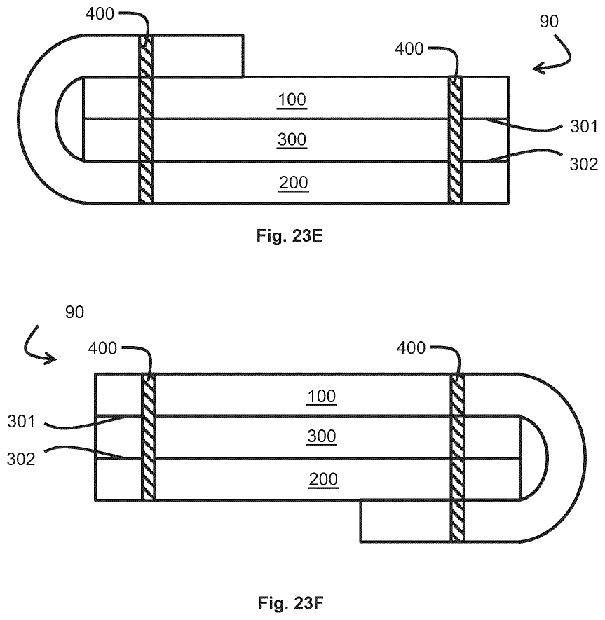

Various elements of the absorbent articles 10 described herein may comprise a stretch laminate. For example, any of the belts 54 and 56 and/or any of the ears 42, 47 may comprise a stretch laminate as described hereinafter. The waistband(s) 36 may also comprise a stretch laminate. Such laminates may include an elastomeric layer that provides extensibility to the laminate and one or more outer layers that is less stretchable but suitable for providing durability and desirable tactile properties. In this way, the laminate permits a component of an absorbent article to closely and comfortably contact the wearer, while providing desirable exterior qualities. FIGS. 23A-F are cross-sectional views of various stretch laminates 90. As shown in FIG. 23A, a stretch laminate 90 may comprise a first cover layer 100 and an elastomeric film layer 300 joined via one or more ultrasonic bonds 400. The elastomeric film layer 300 may have one or more skins, such as a first skin 301 providing first surface, and a second skin 302 providing second surface. As shown in FIG. 23B, a stretch laminate 90 may comprise a first cover layer 100 and a second cover layer 200 with an elastomeric film layer 300 sandwiched therebetween in a facing relationship to both the first cover layer 100 and the second cover layer 200. All three layers may be joined via one or more ultrasonic bonds 400. As shown in FIGS. 23C and 23D, all, or a portion of, the first cover layer 100 may comprise one or more layers, such as a first layer 101 and second layer 102, which may have the same composition or different compositions. Similarly, all or a portion of, the second cover layer 200 may comprise one or more layers, such as a first layer 201 and second layer 202, which may have the same composition or different compositions. As shown in FIGS. 23E and 23F, a portion of the first cover layer 100 or the second cover layer 200 may be folded over to provide a multi-layered structure on all or a portion of the opposite side of stretch laminate 90.

Elastomeric film layer 300 of stretch laminate 90 may include a single layer or multiple layers of one or more materials that are elastically extensible. The elastically extensible material(s) may be between about 10 μm and about 100 μm, or between about 20 μm and about 60 μm, or between about 30 μm and about 50 μm, or in some embodiments, about 40 μm, in thickness. The elastically extensible material(s) may comprise an elastomeric polyolefin, and in some embodiments, a polyolefin (POE) blown film.

The elastically extensible material may comprise modifying resins. The elastically extensible material may comprise a variety of additives. Suitable additives including, but are not limited to, stabilizers, antioxidants, and bacteriostats may be employed to prevent thermal, oxidative, and bio-chemical degradation of the elastically extensible material. Additives may account for about 0.01% to about 60% of the total weight of the elastically extensible material. In other embodiments, the composition comprises from about 0.01% to about 25%. In other suitable embodiments, the elastically extensible material comprises from about 0.01% to about 10% by weight, of additives.

The ultrasonic bonds 400 preferably eliminate the need for any adhesives, but adhesives may be employed to join the layers 100, 200, 300 of the stretch laminate 90. Adhesives may be selected from any adhesives known to provide suitable attachment between elastomeric film layer 300 and cover layers 100, 200. In some embodiments, the adhesive may be a hot melt adhesive with a basis weight of less than about 15 gsm.

Elastomeric film layer 300 may be mechanically pre-activated before attachment to at least one cover layer 100, 200. For example, elastomeric film layer 300 may be pre-activated by being stretched transversely to its web direction by more than 50% (i.e., strain >50%). In some embodiments, an expansion by about 100% to about 500% occurs in relation to the starting width of elastomeric film layer 300. In alternate embodiments, elastomeric film layer 300 may be stretched in the web direction, stretched a direction other than the web direction or transverse to the web direction, or a combination of directions. The term “stretching” is to point to the fact that the expansion of elastomeric film layer 300 is not completely reversible and that a non-elastic fraction results in the film having a larger width following pre-activation (i.e., the elastomeric film does not have 100% recovery, and therefore has a percent set value). After expansion, elastomeric film layer 300 retracts and has a width that may be larger by about 10% to about 30% in relation to a starting width of the film. In other words, after the pre-activation expansion and retraction detailed below, elastomeric film layer 300 may exhibit a set of about 10% to about 30%.

According to various embodiments in which elastomeric film layer 300 includes both an elastically extensible material and at least one skin disposed on the elastically extensible material, the pre-activation process may physically alter these materials differently, for example, because these materials have different elasticity and recovery properties. During pre-activation, the skin 301 and/or 302 and the elastically extensible material are similarly stretched (i.e., put under similar strain). However, after stretching, the skin and the elastically extensible material will retract and recover differently (i.e., have different set values). In comparison with the elastically extensible material, the skin is less elastic and therefore will have less recovery after stretching, a.k.a., a higher set value. The skin is also much thinner than the elastically extensible material, so when the thicker elastically extensible material retracts and recovers after pre-activation stretching, it will force the attached skin to retract with it. But because the skin cannot recover as much as the elastically extensible material, the skin buckles and wrinkles. Accordingly, the cross-sectional profile and the top view appearance of elastomeric film layer 300 are modified after a pre-activation process.

In some configurations, it may be desirable to provide a stretch laminate or an element of an absorbent article 10 (such as a belt or an ear) comprising a stretch laminate with different zones of performance characteristics. Such enhanced properties may include breathability, softness, strength, thickness, uniformity in rugosities, modulus, aesthetic enhancements, tear resistance, combinations of any of the foregoing and/or zones having differing values of any of the foregoing features. According to various embodiments, the stretch laminates and/or elements comprising stretch laminates may comprise: embossing, apertures, perforations, slits, melted material or coatings, compressed material, secondary bonds that are disposed apart from the chassis attachment bond, plastic deformation, and folds.

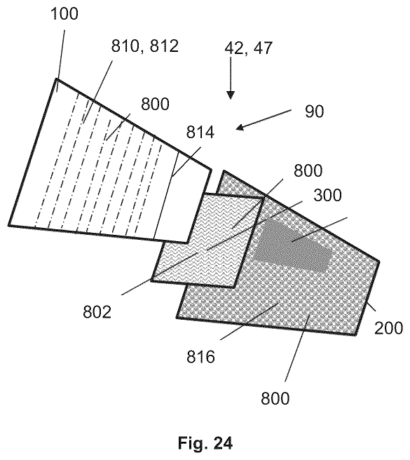

FIG. 24 is an exploded perspective view of an exemplary ear 42, 47 comprising a stretch laminate 90 schematically illustrating exemplary surface modifications. The surface modification(s) may be made to a precursor material prior to lamination. One or more surface modifications 800 (also referred to as morphological features) are illustrated in one or more layers of the laminate. Surface modifications 800 may comprise embossing 802, cuts (e.g., apertures, perforations, slits), melted material or coatings, compressed material 808, plastic deformation 810 (e.g., activation stripes 812), folds 814, post-formation bonds 816 (e.g., adhesive bonds, pressure bonds, thermal bonds, and/or ultrasonic bonds applied after the substrate is formed) and combinations thereof. Surface modifications may be formed after the initial formation of the substrate itself, thereby forming a modified substrate. In other words, thermal bonding fibers to produce a nonwoven is not considered a surface modification. Creating bonds on the nonwoven once it has been formed is considered a surface modification. Additionally, or alternatively, surface modifications 800 on different layers may work together to provide an enhanced property.

One or more structural features may be formed on the laminate after initial bonding of the layers. Structural features may comprise embossing, cuts (e.g., apertures, perforations, slits), melted material or coatings, compressed material, plastic deformation (e.g., activation stripes), folds, secondary bonds (e.g., adhesive bonds, pressure bonds, thermal bonds, and/or ultrasonic bonds applied after the initial bonding of the laminate) and combinations thereof. A structural feature or a surface modification may be in the form a design element.

It is to be appreciated that combinations of one or more surface modifications and one or more structural features may be employed. It is also to be appreciated that certain cover layer substrates may be selected for their amenability to surface modifications and/or structural features.

By way of nonlimiting example, a carded nonwoven may be selected if mechanically activating the substrate and/or laminate. Lower modulus materials, such as polyethylene-based materials, may be more suitable for modification through laser energy.

### Packages

The absorbent articles of the present disclosure may be placed into packages. The packages may comprise polymeric films and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate number of absorbent articles per package. By packaging the absorbent articles under compression, caregivers can easily handle and store the packages, while also providing distribution savings to manufacturers owing to the size of the packages.

### Arrays

“Array” means a display of packages comprising disposable absorbent articles of different article constructions (e.g., different elastomeric materials [compositionally and/or structurally] in the side panels, side flaps and/or belts flaps, different graphic elements, different product structures, fasteners or lack thereof). The packages may have the same brand and/or sub-brand and/or the same trademark registration and/or having been manufactured by or for a common manufacturer and the packages may be available at a common point of sale (e.g., oriented in proximity to each other in a given area of a retail store). An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Huggies,” and same sub-brand, for example, “Pull-Ups.” A different product in the array may have the same brand “Huggies” and the sub-brand “Little Movers.” The differences between the “Pull-Ups” product of the array and the “Little Movers” product in the array may include product form, application style, different fastening designs or other structural elements intended to address the differences in physiological or psychological development. Furthermore, the packaging is distinctly different in that “Pull-Ups” is packaged in a predominately blue or pink film bag and “Little Movers” is packaged in a predominately red film bag.

Further regarding “Arrays,” as another example an array may be formed by different products having different product forms manufactured by the same manufacturer, for example, “Kimberly-Clark”, and bearing a common trademark registration for example, one product may have the brand name “Huggies,” and sub-brand, for example, “Pull-Ups.” A different product in the array may have a brand/sub-brand “Good Nites” and both are registered trademarks of The Kimberly-Clark Corporation and/or are manufactured by Kimberly-Clark. Arrays also often have the same trademarks, including trademarks of the brand, sub-brand, and/or features and/or benefits across the line-up. “On-line Array” means an “Array” distributed by a common on-line source.

### Sanitary Napkin

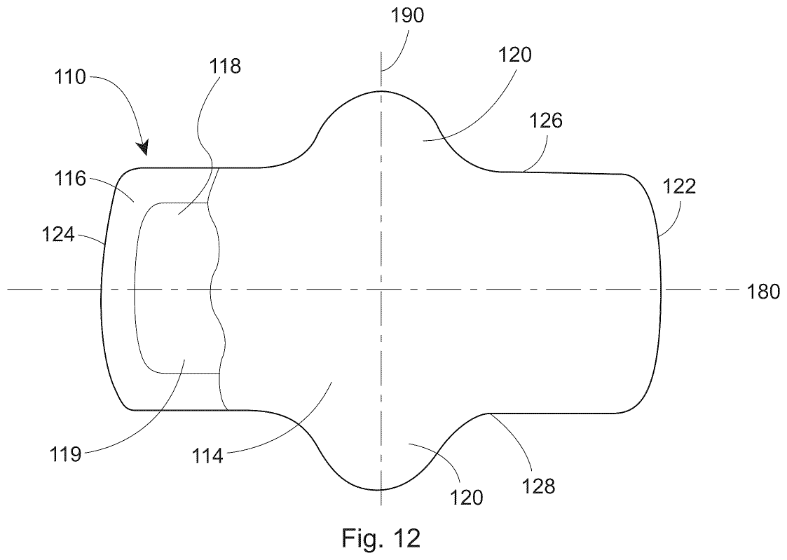

Referring to FIG. 12, an absorbent article of the present disclosure may be a sanitary napkin 110. The sanitary napkin 110 may comprise a liquid permeable topsheet 114, a liquid impermeable, or substantially liquid impermeable, backsheet 116, and an absorbent core 118. The liquid impermeable backsheet 116 may or may not be vapor permeable. The absorbent core 118 may have any or all of the features described herein with respect to the absorbent core 30 and, in some forms, may have a secondary topsheet 119 (STS) instead of the acquisition materials disclosed above. The STS 119 may comprise one or more channels, as described above (including the embossed version). In some forms, channels in the STS 119 may be aligned with channels in the absorbent core 118. The sanitary napkin 110 may also comprise wings 120 extending outwardly with respect to a longitudinal axis 180 of the sanitary napkin 110. The sanitary napkin 110 may also comprise a lateral axis 190. The wings 120 may be joined to the topsheet 114, the backsheet 116, and/or the absorbent core 118. The sanitary napkin 110 may also comprise a front edge 122, a back edge 124 longitudinally opposing the front edge 122, a first side edge 126, and a second side edge 128 longitudinally opposing the first side edge 126. The longitudinal axis 180 may extend from a midpoint of the front edge 122 to a midpoint of the back edge 124. The lateral axis 190 may extend from a midpoint of the first side edge 128 to a midpoint of the second side edge 128. The sanitary napkin 110 may also be provided with additional features commonly found in sanitary napkins as is known in the art.

### Barrier Leg Cuffs

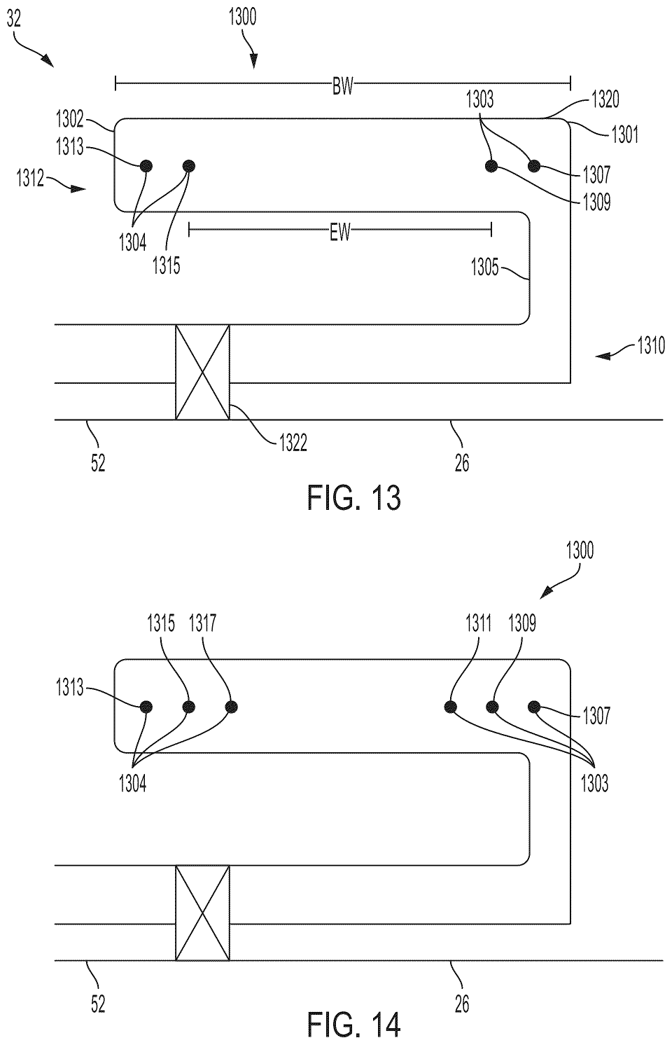

Referring to FIGS. 1 and 2, for example, the absorbent articles 10 of the present disclosure may comprise a pair of barrier leg cuffs 32. The barrier leg cuffs 32 may extend at least partially, or completely, between the front end edge 18 and the back end edge 20 of the absorbent article 10 on opposite sides of the central longitudinal axis 50 and may be at least present in the crotch region 14. Each barrier leg cuff 32 of the pair of barrier leg cuffs may be formed from a single web of material. As shown in FIG. 13, at least a portion of a proximal portion 1310 of the barrier leg cuff 32 may be joined directly or indirectly to the chassis 52. An inner distal portion 1312 of the barrier leg cuff 32 may be opposite the proximal portion 1310 and may be configured to contact the skin of a wearer during use of the absorbent article 10. As such, at least a portion of, or all of, the inner distal portion 1312 of the barrier leg cuff 32 may be unattached to the chassis 52. For example, at least a portion of the inner distal portion 1312 of the barrier leg cuff 32 disposed in the crotch region 14 of the absorbent article 10 may be unattached to the chassis 52, thus allowing the inner distal portion 1312 of the barrier leg cuff 32 to remain in contact with the body of a wearer during wear—especially when the wearer is in motion. The web of material forming the barrier leg cuff 32 may be folded laterally outward to form a backfold 1305 between the proximal portion 1310 and the inner distal portion 1312 of the barrier leg cuff 32. The creation of the backfold 1305 results in the formation of a backfolded inner cuff 1300. The web of material may be further folded laterally inward to form a backfold folded edge 1302 and laterally outward to form an inner folded edge 1301. The backfold folded edge 1302 may be disposed laterally outboard of the inner folded edge 1301 when the absorbent article is in a flat, uncontracted configuration.

As shown in FIG. 13, the inner folded edge 1301 and the backfold folded edge 1302 may be spaced apart from each other such that such that a broad inner face 1320 of the backfolded inner cuff 1300 is formed. This inner face 1320 of the backfolded inner cuff 1300 may be configured to face the body of the wearer while the absorbent article 10 is being worn. A distance (BW) between the inner folded edge 1301 and the backfold folded edge 1302 forming the inner face 1320 of the backfolded inner cuff 1300 may be between about 8 mm and about 35 mm, between about 10 mm and about 30 mm, between about 12 mm and about 25 mm, or between about 15 mm and about 20 mm, specifically reciting all values within these ranges and any ranges formed therein or thereby. A backfolded inner cuff 1300 having a broad inner face 1320 as described herein may reduce the incidence of leakage of bodily exudates from the leg cuff 32 of an absorbent article by creating a relatively wide gasketing region between the leg cuff 32 region of the absorbent article and skin of the wearer. This wide gasketing region may increase the probability that at least a portion of the barrier leg cuff 32 remains in contact with the wearer during use of the absorbent article. A backfolded inner cuff 1300 having a broad inner face 1320, as discussed above, may also reduce the incidence of pressure marking on the skin of the wearer. Pressure marking generally occurs when a concentrated force, for example the force of an elastic of an absorbent article, presses against the skin of a wearer. A backfolded inner cuff 1300 of the present disclosure having a broad inner face 1320 may reduce or eliminate the incidence of pressure marking due, in part, to the forces of the elastics being spread across the broad inner face 1320 instead of being concentrated in a narrower portion of the cuff.

Various portions of the barrier leg cuff 32 may be joined directly or indirectly to a portion of the chassis 52 by a chassis attachment bond 1322. As discussed above, at least a portion of the proximal portion 1310 of the barrier leg cuff 32 may be joined to the chassis 52. A portion of the proximal portion 1310 of the barrier leg cuff 32 may be joined to the chassis from the front end edge 18 to the back end edge 20 of the absorbent article 10 by the chassis attachment bond 1322. The barrier leg cuff 32 may be joined to the chassis 52 by adhesives, thermal bonding, pressure bonding, ultrasonic bonding, combinations thereof, or any other bonding method known in the art. The joinder of the barrier leg cuff 32 to the chassis 52 may be continuous, discontinuous, and/or intermittent. A portion of the barrier leg cuff 32—such as a portion of the proximal portion 1310 of the barrier leg cuff 32—may be joined to the topsheet 26 and/or the backsheet 28.

The backfolded inner cuff 1300 may comprise an inner elastic element 1303 disposed proximate the inner folded edge 1301 and a backfold elastic element 1304 disposed proximate the backfold folded edge 1302. The inner elastic element 1303 and the backfold elastic element 1304 may each comprise a single elastic member or may each comprise a plurality of elastic members. The inner elastic element 1303 and the backfold elastic element 1304 may extend the entire length of each backfolded inner cuff 1300. For example, where the backfolded inner cuff 1300 extends from the front end edge 18 to the back end edge 20 of the absorbent article 10, the inner elastic element 1303 and backfold elastic element 1304 may likewise extend the length of the absorbent article between the front end edge 18 and the back end edge 20. The inner elastic element 1303 and the backfold elastic element 1304 may not extend the entire length of each backfolded inner cuff 1300. The inner elastic element 1303 and the backfold elastic element 1304 may extend only a partial length of the backfolded inner cuff 1300, for example only in the crotch region 14, or only in the crotch region 14 and the back waist region 16. The inventors have found that the backfolded inner cuff 1300 of the present disclosure, comprising two different elastic elements (the inner elastic element and the backfolded elastic element) spaced apart from each other, may form two separate gasketing points between the inner cuff and the body of the wearer, resulting in reduced leaking of bodily exudates out of the absorbent article during wear. In order to create two distinct gasketing points within a single backfolded inner cuff, a distance (EW) between the inner elastic element 1303 and the backfold elastic element 1304 may be between about 5 mm and about 15 mm, between about 6 mm and about 12 mm, or between about 8 mm and about 10 mm, specifically reciting all values within these ranges and any ranges formed therein or thereby.

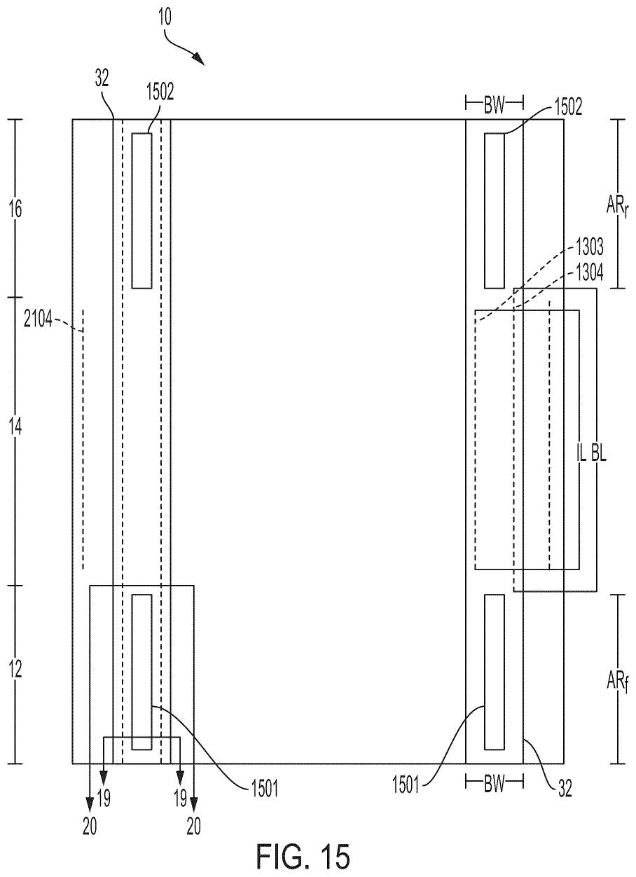

The inner elastic element 1303 and the backfold elastic element 1304 may be attached to the backfolded inner cuff 1300 along the entire length of the backfolded inner cuff 1300, or the elastic elements 1303, 1304 may be attached to only a portion of the backfolded inner cuff 1300. The inner elastic element 1303 and the backfold elastic element 1304 may be attached to the backfolded inner cuff 1300 by any suitable means, such as with adhesive, thermal bonding, pressure bonding, ultrasonic bonding, and combinations thereof. The backfold elastic element 1304 may be attached to the web of material forming the backfolded inner cuff 1300 for a greater longitudinal length as compared to the inner elastic element 1303. Referring to FIG. 15, the backfold elastic element 1304 may have a backfold elastic longitudinal length of attachment (BL), wherein the backfold elastic element 1304 is attached to the web of material forming the backfolded inner cuff 1300 for a longitudinal length (BL) that is between about 1 mm and about 20 mm, between about 2 mm and about 15 mm, or between about 3 mm and about 10 mm greater than an inner elastic element 1303 length of attachment (IL), specifically reciting all values within these ranges and any ranges formed therein or thereby. A greater backfold elastic longitudinal length of attachment (BL) as compared to an inner elastic element length of attachment (IL) is believed to result in a wear configuration in which both the backfold folded edge 1302 and the inner folded edge 1301 of the backfolded inner cuff 1300 maintain contact with the skin of the wearer during use.

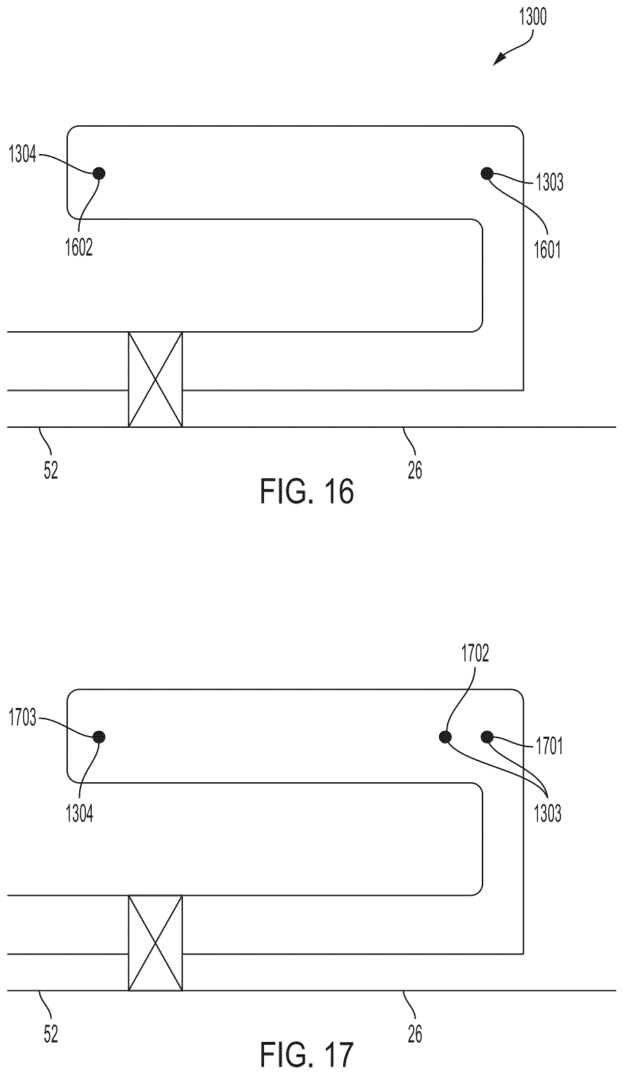

The inner elastic element 1303 may comprise a single elastic member (as shown in FIG. 16) or may comprise a plurality of elastic members (as shown in FIGS. 13, 14, and 17). The inner elastic element 1303 may comprise a first inner elastic member 1307. The inner elastic element 1303 may comprise a second inner elastic member 1309. The inner elastic element 1303 may comprise a third inner elastic member 1311. The first 1307, second 1309, and third 1311 inner elastic members may all be the same material, or they may be different materials. For example, the first 1307, second 1309, and third 1311 inner elastic members may all be elastic strands of the same or different linear densities.

The backfold elastic element 1304 may comprise a single elastic member (as shown in FIG. 16) or may comprise a plurality of elastic members (as shown in FIGS. 13, 14, and 18). The backfold elastic element 1304 may comprise a first backfold elastic member 1313. The backfold elastic element 1304 may comprise a second backfold elastic member 1315. The backfold elastic element 1304 may comprise a third backfold elastic member 1317. The first 1313, second 1315, and third 1317 backfold elastic members may all be the same material, or they may be different material. For example, the first 1313, second 1315, and third 1317 backfold elastic members may all be elastic strands of the same or different linear densities.

Regardless of the composition of the inner elastic element 1303 and the backfold elastic element 1304 (whether there is a single elastic member or a plurality of elastic members, for example), the inner elastic element 1303 may be configured to impart a different contraction force as compared to the backfold elastic element 1304. The inner elastic element 1303 may be configured to impart a greater contraction force as compared to the backfold elastic element 1304. The inventors have found that a backfolded inner cuff 1300 having a difference in contraction force between the elastic element proximate the inner folded edge 1301 and the elastic element proximate the backfold folded edge 1302 may allow the backfolded inner cuff 1300 to better conform to the contours of the wearer during use of the absorbent article, thereby ensuring that both the region of the backfolded inner cuff 1300 proximate the inner folded edge 1301 and the region proximate the backfold folded edge 1302 remain in contact with the body of the wearer during use. Maintenance of contact between portions of the backfolded inner cuff 1300 and the body of the wearer—especially while the wearer is in motion and the contours of the body may change—allows the inner cuff gasket to be maintained and reduces or prevents leakage of bodily exudates.

The backfold elastic element 1304 may be configured to impart a total contraction force that is between about 30% and about 95%, between 45% and about 90%, or between about 60% and about 85% of a total contraction force imparted by the inner elastic element 1303, specifically reciting all values within these ranges and any ranges formed therein or thereby. The total contraction force imparted by the inner elastic element 1303 and the backfold elastic element 1304 may be created by any method known in the art, including the selection of elastic members of varying linear densities, elastic pre-straining (straining an elastic element prior to attachment to the web of material forming the barrier leg cuff), and the like.

Regardless of whether the inner elastic element 1303 and the backfold elastic element 1304 are formed from a single elastic member or a plurality of elastic members, the inner elastic element 1303 may have a total linear density that is greater than a total linear density of the backfold elastic element 1304. The inner elastic element 1303 may have a total linear density of between about 800 dtex and about 1500 dtex, between about 850 dtex and about 1450 dtex, between about 900 dtex and about 1400 dtex, or between about 940 dtex and about 1360 dtex, specifically reciting all values within these ranges and any ranges formed therein or thereby. Where the inner elastic element 1303 comprises a plurality of elastic members, the individual linear densities of each elastic member are added together to result in a total linear density. The backfold elastic element 1304 may have a total linear density of between about 240 dtex and about 1425 dtex, between about 255 dtex and about 1378 dtex, between about 270 dtex and about 1330 dtex, or between about 282 dtex and about 1295 dtex, specifically reciting all values within these ranges and any ranges formed therein or thereby. Where the backfold elastic element 1304 comprises a plurality of elastic members, the individual linear densities of each elastic member are added together to result in a total linear density. The individual elastic members that comprise both the inner elastic element 1303 and the backfold elastic element 1304 may each have a linear density between about 350 dtex and about 1200 dtex, between about 400 dtex and about 1150 dtex, or between about 500 dtex and about 1000 dtex, specifically reciting all values within these ranges and any ranges formed therein or thereby.

Regardless of whether the inner elastic element 1303 and the backfold elastic element 1304 are formed from a single elastic member or a plurality of elastic members, the inner elastic element 1303 may have a total pre-strain that is greater than a total pre-strain of the backfold elastic element 1304. The inner elastic element 1303 may have a pre-strain of between about 150% and about 350%, between about 175% and about 325%, or between about 200% and about 300%, specifically reciting all values within these ranges and any ranges formed therein or thereby. The backfold elastic element 1304 may have a pre-strain of between about 150% and about 350%, between about 175% and about 325%, or between about 200% and about 300%, specifically reciting all values within these ranges and any ranges formed therein or thereby. Where the inner elastic element 1303 and/or the backfold elastic element 1304 comprise a plurality of elastic members, the pre-strain may be a measure of the pre-strain of an individual elastic members as opposed to a total pre-strain of all of the elastic members. All the elastic members comprising the inner elastic element 1303 may be configured to have the same pre-strain. Likewise, all the elastic members comprising the backfold elastic element 1304 may be configured to have the same pre-strain.

Referring again to FIG. 13, the inner elastic element 1303 may be formed from a plurality of inner elastic members 1307, 1309, and the backfold elastic element 1304 may be formed from a plurality of backfold elastic members 1313, 1315. In a form, the plurality of inner elastic members 1307, 1309 may each have the same linear density and be configured to have the same pre-strain. In another form, the plurality of inner elastic members 1307, 1309 may have different linear densities and be configured to have different pre-strain. In another form, the plurality of inner elastic members 1307, 1309 may each have the same linear density and be configured to have different pre-strain. In yet another form, the plurality of inner elastic members 1307, 1309 may each have different linear densities and be configured to have different pre-strain.

Still referring to FIG. 13, the plurality of backfold elastic members 1313, 1315 may each have the same linear density and be configured to have the same pre-strain. In another form, the plurality of backfold elastic members 1313, 1315 may have different linear densities and be configured to have different pre-strain. In another form, the plurality of backfold elastic members 1313, 1315 may each have the same linear density and be configured to have different pre-strain. In yet another form, the plurality of backfold elastic members 1313, 1315 may each have different linear densities and be configured to have different pre-strain. The inner elastic element 1303 and backfold elastic element 1304 may each comprise more than two elastic members, for example as shown in FIG. 14, where the inner elastic element 1303 and backfold elastic element 1304 each are formed from three elastic members.

Referring to FIG. 16, the inner elastic element 1303 may be formed from a single inner elastic member 1601, and the backfold elastic element 1304 may be formed from a single backfold elastic member 1602. In a form, the single inner elastic member 1601 and the single backfold elastic member 1602 may have the same liner density and be configured to have different pre-strain. The inner elastic member 1601 pre-strain may be greater than the single backfold elastic member 1602 pre-strain. In another form, the single inner elastic member 1601 and the single backfold elastic member 1602 may be configured to have the same pre-strain and have different linear densities. The single inner elastic member 1601 linear density may be greater than the single backfold elastic member 1602 linear density.

The inner elastic element 1303 may comprise a different number of inner elastic members as compared to the number of backfold elastic members that comprise the backfold elastic element 1304. Referring to FIG. 17, the inner elastic element 1303 may comprise a plurality of inner elastic members—as shown in FIG. 17 a first inner elastic member 1701 and a second inner elastic member 1702. The backfold elastic element 1304 may comprise a single backfold elastic member 1703. As discussed above, the individual elastic elements 1701, 1702, and 1703 may have the same linear density or different linear densities. Likewise, the individual elastic elements 1701, 1702, and 1703 may be configured to have the same pre-strain or may be configured to have different pre-strains. The inner elastic element 1303 may be configured to have a total contraction force greater than the total contraction force of the backfold elastic element 1304.

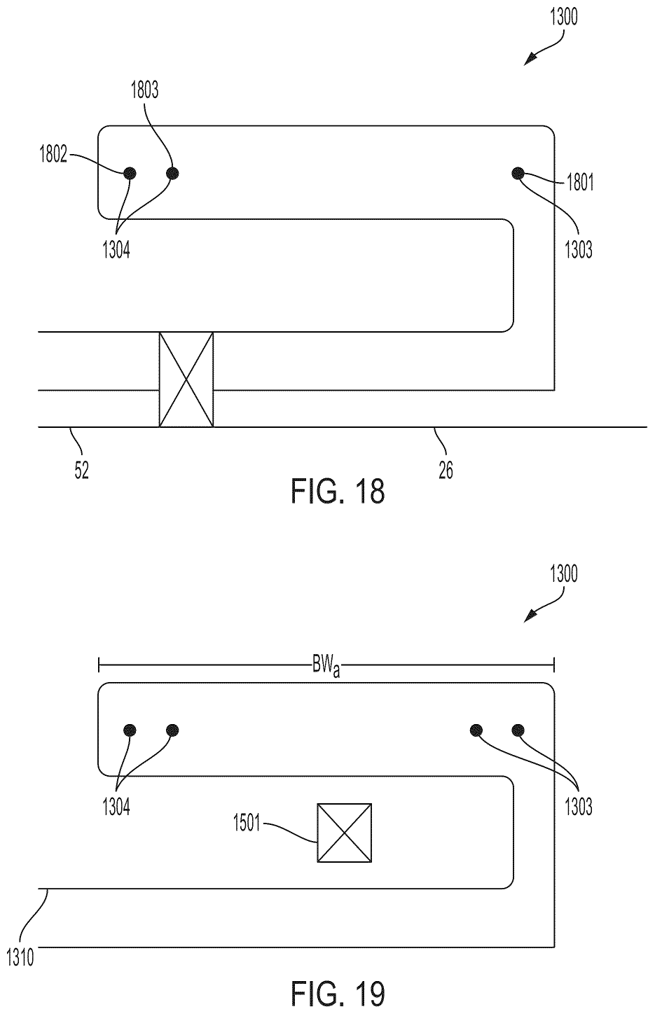

Referring now to FIG. 18, the inner elastic element 1303 may comprise a single elastic member 1801, and the backfold elastic element 1304 may comprise a plurality of elastic members—1802 and 1803 as shown in FIG. 18. As discussed above, the individual elastic elements 1801, 1802, and 1803 may have the same linear density or different linear densities. Likewise, the individual elastic elements 1801, 1802, and 1803 may be configured to have the same pre-strain or may be configured to have different pre-strains. The inner elastic element 1303 may be configured to have a total contraction force greater than the total contraction force of the backfold elastic element 1304.

The barrier leg cuffs 32 may be configured such that at least a portion of, or all of, the backfolded inner cuff 1300 may overlap the topsheet 24 when the absorbent article is in a flat, uncontracted configuration. It is believed that partial or complete overlap of the backfolded inner cuff 1300 and the topsheet 26 may result in a reduced incidence of leakage of bodily exudate from the absorbent article. It is believed that maintenance of the backfolded inner cuff 1300 proximate to the permeable topsheet 26 provides a greater likelihood that the bodily exudate will be wicked through the topsheet 26 and be locked away in the absorbent core, rather than escaping out the side of the article.

At least a portion of the backfolded inner cuff 1300 may be joined to another portion of the web of material forming the barrier leg cuff 32. Referring to FIG. 15, a portion of the backfolded inner cuff 1300 disposed in the front waist region 12 of the absorbent article 10 may be joined to a portion of the web of material forming the barrier leg cuff 32 by a first inner cuff attachment bond 1501. A portion of the backfolded inner cuff 1300 disposed in the back waist region 16 of the absorbent article 10 may be joined to a portion of the web of material forming the barrier leg cuff 32 by a second inner cuff attachment bond 1502. As can be seen in the cross-sectional view of a portion of a barrier leg cuff 32 disposed in the front waist region 12 in FIG. 19, a portion of the backfolded inner cuff 1300 formed by the inner distal portion 1312 of the web of material forming the barrier leg cuff 32 is attached to the proximal portion 1310 of the web of material by a first inner cuff attachment bond 1501. Said differently, at least a portion of the backfolded inner cuff 1300 between the inner folded edge 1301 and the backfold folded edge 1302 may be joined to a portion of the web of material disposed between the backfolded inner cuff 1300 and the topsheet 26 by a first inner cuff attachment bond 1501 and/or a second inner cuff attachment bond 1502. Furthermore, a portion of the web of material forming the backfolded inner cuff 1300 and between the inner folded edge 1301 and the backfold folded edge 1302 may be joined to a portion of the web of material between the proximal portion 1310 of the barrier leg cuff and the backfold 1305 by a first inner cuff attachment bond 1501 and/or a second inner cuff attachment bond 1502.

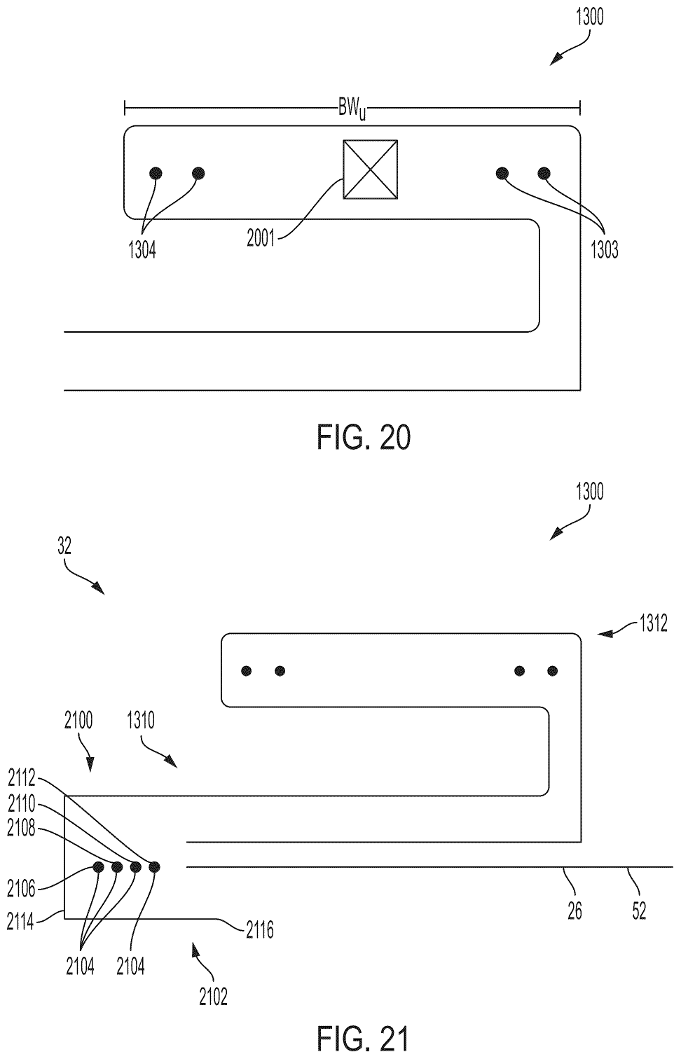

The portions of the barrier leg cuff 32 comprising inner cuff attachment bonds 1501, 1502 may define attachment region(s). As shown in FIG. 15, a front attachment region (AR_{f}) may be disposed in the front waist region 12, and a rear attachment region (ARᵣ) may be disposed in the rear waist region 14. The front attachment region and the rear attachment region may be separated by a region of the backfolded inner cuff 1300 that is unattached to another portion of the web of material forming the barrier leg cuff 32, which may be disposed primarily in the crotch region 14 of the absorbent article 10. FIGS. 19 and 20 show cross-sectional views of the attachment region (ARf, ARr) and the unattached region. When the absorbent article 10 is in a flat, uncontracted state, the portion of the backfolded inner cuff disposed in an attachment region (AR_{f}, ARᵣ) may have an attachment region wearer-facing surface width (Bwₐ), and the portion of the backfolded inner cuff disposed outside an attachment region, such as in the crotch region 14, may have an unattached region wearer-facing surface width (BWᵤ). The attachment region wearer-facing surface width (Bwₐ) may be greater than the unattached region wearer-facing surface width (BWᵤ). The attachment region wearer-facing surface width (Bwₐ) may be between about 0.5 mm and about 5 mm greater, between about 1 mm and about 3.5 mm greater, or about 2 mm greater than the unattached region wearer-facing surface width (BWᵤ), specifically reciting all values within these ranges and any ranges formed therein or thereby.

Inner cuff attachment bonds 1501, 1502 may be by adhesives, thermal bonding, pressure bonding, ultrasonic bonding, combinations thereof, or any other bonding method known in the art. The inner cuff attachment bonds 1501, 1502 may be continuous, discontinuous, and/or intermittent.

As shown in FIG. 20, the backfolded inner cuff 1300 may comprise a hem bond 2001 disposed within the backfolded inner cuff 1300 and between the inner elastic element 1303 and the backfold elastic element 1304. The hem bond may separate the inner elastic element 1303 and the backfold elastic element 1304 within the backfolded inner cuff 1300. The hem bond 2001 may be formed by adhesives, thermal bonding, pressure bonding, ultrasonic bonding, combinations thereof, or any other bonding method known in the art. The hem bond may be continuous, discontinuous, and/or intermittent. Without wishing to be bound by theory, it is believed that the hem bond 2001 may add rigidity to the backfolded inner cuff 1300, allowing for greater stability and greater control during attachment of the barrier leg cuffs 32 to the absorbent article. The increased rigidity from the presence of the hem bond 2001 may allow for easier and more accurate placement of the barrier leg cuffs 32 onto the absorbent article 10.

Referring to FIG. 21, the web of material forming the barrier leg cuffs 32 may form an outer cuff 2100 in addition to forming the backfolded inner cuff 1300 as discussed herein. The outer cuff 2100 may comprise an outer cuff folded edge 2114 and an outer cuff material edge 2116. The web of material forming the barrier leg cuff 32 may be folded laterally inward to form the outer cuff folded edge 2114. The outer cuff 2100 may form an outer distal portion 2102 of the barrier leg cuff 32, disposed laterally outboard of the proximal portion 1310 of the barrier leg cuff 32 when the absorbent article 10 is in a flat, uncontracted state. The outer cuff 2100 may be disposed laterally outboard of the topsheet 26 when the absorbent article 10 is in a flat, uncontracted state.

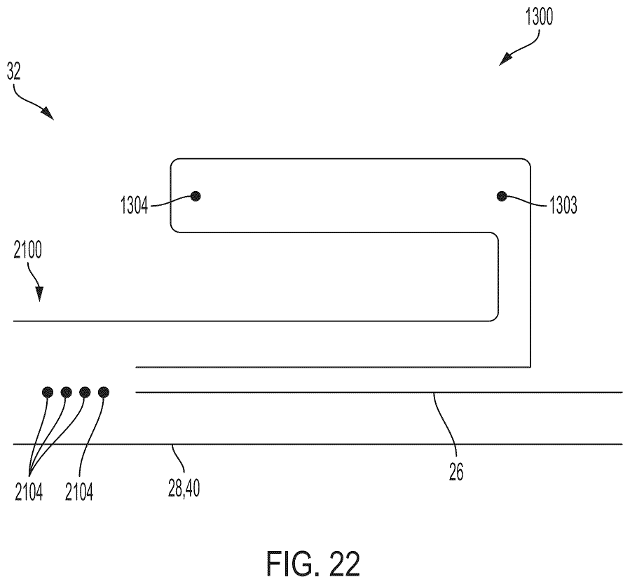

The outer cuff 2100 may comprise an outer cuff elastic element 2104. The outer cuff elastic element 2104 may be disposed proximate the outer cuff folded edge 2114. The outer cuff elastic element 2104 may be formed from a single elastic member 2106, or may be formed from a plurality of elastic members, for example 2106, 2108, 2110, and 2110 as shown in FIG. 21. The outer cuff elastic element 2104 may extend at least partially between the front end edge 18 and the back end edge 20 of the absorbent article 10. The outer cuff elastic element 2104 may be joined to the web of material forming the barrier leg cuff 32 at least in the crotch region 14 of the absorbent article. The outer cuff elastic element 2104 may be disposed between two surfaces of the web of material forming the barrier leg cuff 32, as shown in FIG. 21. As shown in FIG. 22, the outer cuff elastic element 2104 may be disposed between the web of material forming the barrier leg cuff 32 and a portion of the backsheet 28. In a form, the outer cuff elastic element 2104 may be disposed between the web of material forming the barrier leg cuff 32 and the backsheet film. In another form, the outer cuff elastic element 2104 may be disposed between the web of material forming the barrier leg cuff 32 and the outer cover material 40.

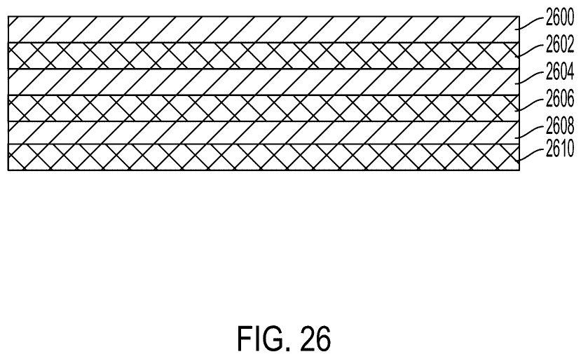

Referring to FIG. 26, an example cross-sectional view of a nonwoven material forming the barrier leg cuff 32 illustrated. The cross-sectional view does not include elastic strands or films. The barrier leg cuff 32 may comprise, in order or not in order, a first spunbond nonwoven layer 2600, a second spunbond nonwoven layer 2602, a third meltblown nonwoven layer 2604, a fourth meltblown nonwoven layer 2606, a fifth meltblown nonwoven layer 2608, and a sixth spunbond nonwoven layer 2610. This can also be known as a SSMMMS nonwoven material, with S being a spunbond nonwoven layer and M being a meltblown nonwoven layer.

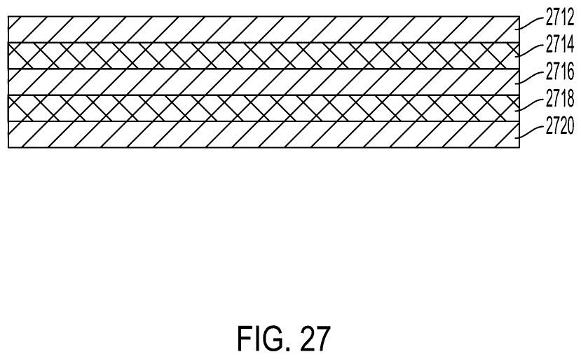

Referring to FIG. 27, another example cross-sectional view of a nonwoven material forming the barrier leg cuff 32 is illustrated. The cross-sectional view does not include elastic strands or films. The barrier leg cuff 32 may comprise, in order or not in order, a first spunbond nonwoven layer 2712, a second spunbond nonwoven layer 2714, a third meltblown nonwoven layer 2716, a fourth meltblown nonwoven layer 2718, a fifth spunbond nonwoven layer 2720. This can also be known as a SSMMS nonwoven material with S being a spunbond nonwoven layer and M being a meltblown nonwoven layer.

### Bio-Based Content for Components

Components of the absorbent articles described herein may at least partially be comprised of bio-based content as described in U.S. Pat. Appl. No. 2007/0219521 A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and 2016/0206774, and U.S. Pat. No. 9,169,366. Example bio-based polyolefins for use in the present disclosure comprise polymers available under the designations SHA7260™, SHE150™, or SGM9450F™ (all available from Braskem S.A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. Appl. Publ. No. 2019/0192723, published on Jun. 27, 2019.

### Combinations

1. An absorbent article (10) comprising:
a liquid permeable topsheet (26);
a liquid impermeable backsheet (28);
an absorbent core (30) disposed at least partially between the topsheet (26) and the backsheet (28); and
a pair of barrier leg cuffs (32), each barrier leg cuff (32) formed from a single web of material and comprising a backfolded inner cuff (1300) comprising:
an inner folded edge (1301),
a backfold folded edge (1302),
an inner elastic element (1303), and
a backfold elastic element (1304);
   wherein the inner elastic element (1303) is disposed proximate the inner folded edge (1301), and the backfold elastic element (1304) is disposed proximate the backfold folded edge (1302); and wherein at least a portion of the backfolded inner cuff (1300) is configured to overlap the topsheet (26) when the absorbent article is in a flat, uncontracted configuration.
   2. The absorbent article (10) of paragraph 1, wherein the web of material is folded laterally outward to form the inner folded edge (1301), and wherein the web of material is folded laterally inward to form the backfold folded edge (1302).
   3. The absorbent article (10) of any of paragraphs 1 or 2, wherein the inner elastic element (1303) comprises a first inner elastic member (1307).
   4. The absorbent article (10) of paragraph 3, wherein the inner elastic element (1303) comprises a second inner elastic member (1309).
   5. The absorbent article (10) of any of paragraphs 3 or 4, wherein the first inner elastic member (1307) and/or the second inner elastic member (1309) is an elastic strand having a linear density between about 350 dtex and about 1200 dtex, between about 400 dtex and about 1150 dtex, or between about 500 dtex and about 1000 dtex.
   6. The absorbent article (10) of any one of the preceding paragraphs, wherein the backfold elastic element (1304) comprises a first backfold elastic member (1313).
   7. The absorbent article (10) of paragraph 6, wherein the first backfold elastic member (1313) is an elastic strand having a linear density between about 350 dtex and about 1200 dtex, between about 400 dtex and about 1150 dtex, or between about 500 dtex and about 1000 dtex.
   8. The absorbent article (10) of any one of the preceding paragraphs, wherein the inner elastic element (1303) is configured to have a first contraction force, wherein the backfold elastic element (1304) is configured to have a second contraction force, and wherein the first contraction force is different than the second contraction force.
   9. The absorbent article (10) of paragraph 8, wherein the first contraction force is greater than the second contraction force.
   10. The absorbent article (10) of any one of the preceding paragraphs, wherein at least a portion of the backfolded inner cuff (1300) between the inner folded edge (1301) and the backfold folded edge (1302) is joined to a portion of the web of material disposed between the backfolded inner cuff (1300) and the topsheet (26).
   11. The absorbent article (10) of any one of the preceding paragraphs, wherein each barrier leg cuff (32) further comprises an outer cuff (2100) comprising an outer cuff folded edge (2114), an outer cuff material end (2116), and an outer cuff elastic element (2104).
   12. The absorbent article (10) of paragraph 11, wherein the web of material is folded laterally inward to form the outer cuff folded edge (2114).
   13. The absorbent article (10) of paragraphs 11 or 12, wherein the outer cuff elastic element (2104) is disposed proximate the outer cuff folded edge (2114).
   14. The absorbent article (10) of any one of paragraphs 11-13, wherein the outer cuff elastic element (2104) comprises a plurality of elastic members.
   15. The absorbent article (10) of any one of the preceding paragraphs, wherein the absorbent article (10) is a disposable insert (2500) configured to engage with a reusable outer cover (2502).
   16. The absorbent article (10) of any one of paragraphs 1-14, wherein the absorbent article (10) is a taped-style diaper comprising a secondary fastening system comprising a secondary fastening component (210) and a secondary receiving component (212).
   17. An absorbent article (10) comprising:
   a chassis (52) comprising a liquid permeable topsheet (26), a liquid impermeable backsheet (28), and an absorbent core (30) disposed at least partially between the topsheet (26) and the backsheet (28); and
   a pair of barrier leg cuffs (32), each barrier leg cuff (32) formed from a single web of material and comprising a proximal portion (1310) joined to the chassis (52) and an inner distal portion (1312), wherein the web of material is folded laterally outward to form a backfold (1305) between the proximal portion (1310) and the inner distal portion (1312), forming a backfolded inner cuff (1300), the backfolded inner cuff (1300) comprising an inner folded edge (1301), a backfold folded edge (1302), an inner elastic element (1303), and a backfold elastic element (1304).
   18. The absorbent article (10) of paragraph 17, wherein a portion of the web of material forming the backfolded inner cuff (1300) and between the inner folded edge (1301) and the backfold folded edge (1302) is joined to a portion of the web of material between the proximal portion (1310) of the barrier leg cuff and the backfold (1305).
   19. The absorbent article (10) of paragraph 17 or 18, wherein the backfolded inner cuff (1300) is configured to overlap the topsheet (26) when the absorbent article is in a flat, uncontracted configuration.
   20. The absorbent article (10) of any one of paragraphs 16-19, wherein the web of material is folded laterally outward to form the backfolded inner cuff (1300) inner folded edge (1301), and wherein the web of material is folded laterally inward to form the backfold folded edge (1302).
   21. The absorbent article (10) of any one of paragraphs 16-20, wherein the inner elastic element (1303) comprises a first inner elastic member (1307).
   22. The absorbent article (10) of paragraph 21, wherein the inner elastic element (1303) comprises a second inner elastic member (1309).
   23. The absorbent article (10) of any of paragraphs 21 or 22, wherein the first inner elastic member (1307) and/or the second inner elastic member (1309) is an elastic strand having a linear density between about 350 dtex and about 1200 dtex, between about 400 dtex and about 1150 dtex, or between about 500 dtex and about 1000 dtex.
   24. The absorbent article (10) of any one of paragraphs 16-23, wherein the backfold elastic element (1304) comprises a first backfold elastic member (1313).
   25. The absorbent article (10) of paragraph 24, wherein the first backfold elastic member (1313) is an elastic strand having a linear density of between about 350 dtex and about 1200 dtex, between about 400 dtex and about 1150 dtex, or between about 500 dtex and about 1000 dtex.
   26. The absorbent article (10) of any one of paragraphs 16-25, wherein the inner elastic element (1303) is configured to have a first contraction force, wherein the backfold elastic element (1304) is configured to have a second contraction force, and wherein the first contraction force is different than the second contraction force.
   27. The absorbent article (10) of paragraph 26, wherein the first contraction force is greater than the second contraction force.
   28. The absorbent article (10) of any one of paragraphs 16-27, wherein at least a portion of the backfolded inner cuff (1300) between the inner folded edge (1301) and the backfold folded edge (1302) is joined to a portion of the web of material disposed between the backfolded inner cuff (1300) and the proximal portion (1310) of the barrier leg cuff (32).
   29. The absorbent article (10) of any one of paragraphs 16-28, wherein each barrier leg cuff (32) further comprises an outer cuff (2100) comprising an outer cuff folded edge (2114), an outer cuff material end (2116), and an outer cuff elastic element (2104).
   30. The absorbent article (10) of paragraph 29, wherein the web of material is folded laterally inward to form the outer cuff folded edge (2114).
   31. The absorbent article (10) of paragraphs 29 or 30, wherein the outer cuff elastic element (2104) is disposed proximate the outer cuff folded edge (2114).
   32. The absorbent article (10) of any one of paragraphs 29-31, wherein the outer cuff elastic element (2104) comprises a plurality of elastic members.
   33. The absorbent article (10) of any one of paragraphs 16-32, wherein the absorbent article (10) is a disposable insert (2500) configured to engage with a reusable outer cover (2502).
   34. The absorbent article (10) of any one of paragraphs 16-32, wherein the absorbent article (10) is a taped-style diaper comprising a secondary fastening system comprising a secondary fastening component (210) and a secondary receiving component (212).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this present disclosure.

What is claimed is:

1. An absorbent article comprising:
a liquid permeable topsheet;
a liquid impermeable backsheet;
an absorbent core disposed at least partially between the topsheet and the backsheet; and
a pair of barrier leg cuffs, each barrier leg cuff formed from a single web of material and comprising a backfolded inner cuff comprising:
an inner folded edge,
a backfold folded edge,
an inner elastic element, and
a backfold elastic element;
wherein the inner elastic element is disposed proximate the inner folded edge, and the backfold elastic element is disposed proximate the backfold folded edge;
wherein the inner elastic element is configured to have a first contraction force, wherein the backfold elastic element is configured to have a second contraction force, and wherein the first contraction force is different than the second contraction force; and
wherein at least a portion of the backfolded inner cuff is configured to overlap the topsheet when the absorbent article is in a flat, uncontracted configuration.

2. The absorbent article of claim 1, wherein the web of material is folded laterally outward to form the inner folded edge, and wherein the web of material is folded laterally inward to form the backfold folded edge.

3. The absorbent article of claim 1, wherein the inner elastic element comprises a first inner elastic member.

4. The absorbent article of claim 3, wherein the inner elastic element comprises a second inner elastic member.

5. The absorbent article of claim 4, wherein the first inner elastic member and/or the second inner elastic member is an elastic strand having a linear density between about 350 dtex and about 1200 dtex.

6. The absorbent article of claim 1, wherein the backfold elastic element comprises a first backfold elastic member.

7. The absorbent article of claim 6, wherein the first backfold elastic member is an elastic strand having a linear density between about 350 dtex and about 1200 dtex.

8. The absorbent article of claim 1, wherein the first contraction force is greater than the second contraction force.

9. The absorbent article of claim 1, wherein at least a portion of the backfolded inner cuff between the inner folded edge and the backfold folded edge is joined to a portion of the web of material disposed between the backfolded inner cuff and the topsheet.

10. The absorbent article of claim 1, wherein each barrier leg cuff further comprises an outer cuff comprising an outer cuff folded edge, an outer cuff material end, and an outer cuff elastic element.

11. The absorbent article of claim 10, wherein the web of material is folded laterally inward to form the outer cuff folded edge.

12. The absorbent article of claims 11, wherein the outer cuff elastic element is disposed proximate the outer cuff folded edge.

13. The absorbent article of any one of claims 10, wherein the outer cuff elastic element comprises a plurality of elastic members.

14. The absorbent article of claim 1, wherein the absorbent article is a disposable insert configured to engage with a reusable outer cover.

15. The absorbent article of claim 1, wherein the absorbent article is a taped-style diaper comprising a secondary fastening system comprising a secondary fastening component and a secondary receiving component.

16. An absorbent article comprising:
a chassis comprising a liquid permeable topsheet, a liquid impermeable backsheet, and an absorbent core disposed at least partially between the topsheet and the backsheet; and
a pair of barrier leg cuffs, each barrier leg cuff formed from a single web of material and comprising a proximal portion joined to the chassis and an inner distal portion, wherein the web of material is folded laterally outward to form a backfold between the proximal portion and the inner distal portion, forming a backfolded inner cuff, the backfolded inner cuff comprising an inner folded edge, a backfold folded edge, an inner elastic element, and a backfold elastic element;
wherein the inner elastic element is configured to have a first contraction force, wherein the backfold elastic element is configured to have a second contraction force, and wherein the first contraction force is different than the second contraction force.

17. The absorbent article of claim 16, wherein the first contraction force is greater than the second contraction force.

18. The absorbent article of claim 16, wherein each barrier leg cuff further comprises an outer cuff comprising an outer cuff folded edge, an outer cuff material end, and an outer cuff elastic element.

19. The absorbent article of claim 18, wherein the web of material is folded laterally inward to form the outer cuff folded edge, and wherein the outer cuff elastic element is disposed proximate the outer cuff folded edge.

20. The absorbent article of claim 18, wherein the outer cuff elastic element comprises a plurality of elastic members.
